(19) United States
(12) Patent Application Publication
(10) Pub. No.: US 2026/0253665 A1
(43) Pub. Date: Aug. 27, 2026

(54) METHODS AND SYSTEMS TO ENHANCE A PLANT BREEDING PIPELINE

(71) Applicant: PIONEER HI-BRED INTERNATIONAL, INC, JOHNSTON, IA (US)

(72) Inventors: ANDREW BAUMGARTEN, JOHNSTON, IA (US); JHONATHAN PEDROSO RIGAL DOS SANTOS, JOHNSTON, IA (US); ELI RODGERS-MELNICK, JOHNSTON, IA (US)

(73) Assignee: PIONEER HI-BRED INTERNATIONAL, INC, JOHNSTON, IA (US)

(21) Appl. No.: 18/877,457

(22) PCT Filed: Jun. 23, 2023
(86) PCT No.: PCT/US2023/068985

§ 371 Date: Dec. 20, 2024

Related U.S. Application Data
(60) Provisional application No. 63/366,990, filed on Jun. 24, 2022.
(60) Provisional application No. 63/494,472, filed on Apr. 6, 2023.

(51) Int. Cl. G16B 20/00; G16B 40/00

(52) U.S. Cl. CPC G16B 20/00; G16B 40/00

(57) ABSTRACT

Systems and methods that use machine learning models or deep learning models to learn a relationship of genotype by environment interactions with the phenotypic performance for a plurality of plant genotypes and predict, for one or more candidate plant genotypes, or between pairs or of candidate plant genotypes, relative performance for a phenotype of interest using genetic and environmental data are provided herein. The systems and methods may be used to identify and select candidate plants for commercial products and targeted breeding for certain environments and locations.

### FIELD

The disclosure relates to the field of plant breeding.

### BACKGROUND

The contribution of plant breeding to agricultural productivity continues to grow at an astronomical rate as plant breeders have been adept at assimilating and integrating information from extensive potential lines and applying advanced breeding approaches to create a breeding pipeline that has continuous population improvement and delivers valued products for farmers, end-users, and consumers.

### SUMMARY

Disclosed herein are methods and systems for use in predicting the performance of a plant or plant genotype. In some embodiments, the methods may include inputting, through one or more computing devices, representations of genotypic data from two or more candidate plant genotypes and location-specific environmental data into a trained machine learning model. In some aspects, the methods include inputting representations of spatial coordinate data where the two or more candidate plant genotypes' performances will be predicted. In some examples, at least one of the two or more candidate plant genotypes is a reference candidate plant genotype. In some examples, the machine learning model has been trained to learn a relationship of genotype by environment interactions with the phenotypic performance for a plurality of plant genotypes, i.e., genotype by environment interactions, and predict performance for a phenotype of interest for one or more of the candidate plant genotypes compared to the reference candidate plant genotype. The methods may include generating by the trained machine model a prediction as to the performance of the one or more candidate plant genotypes for the phenotype of interest as compared to the reference candidate plant genotype.

In some embodiments, the systems may include one or more servers and a computing device communicatively coupled to the one or more servers. One or more of the servers may include representations of genotypic data and location-specific environmental data associated with two or more candidate plant genotypes. The computing device may include a memory and one or more processors configured to perform operations. In some examples, the one or more processors is configured to obtain representations of genotypic data for two or more candidate plant genotypes and associated location-specific environmental data at one or more locations. In some examples, the one or more processors is configured to generate, using a machine learning model, a prediction as to the performance for one or more candidate plant genotypes for a phenotype of interest as compared to a reference candidate plant genotype at one or more locations.

In some embodiments, the systems may include one or more servers and a computing device communicatively coupled to the one or more servers. One or more of the servers may include representations of genotypic data, location-specific environmental data, and spatial coordinate data from two or more candidate plant genotypes and representations of genotypic data, location-specific environmental data, and spatial data associated with two or more training plants. The computing device may include a memory and one or more processors configured to perform operations. In some examples, the one or more processors is configured to obtain at least one training data set comprising representations of genotypic data, location-specific environmental data, and spatial data associated with two or more training plants at one or more locations. In some examples, the one or more processors is configured to simultaneously learn from the training data set an association among training plant genotypes and location-specific environment interactions, e.g., the relationship of genotype by environment interactions with the phenotypic performance for a plurality of plant genotypes, to generate a performance prediction for a phenotype of interest for one or more plant genotypes at one or more locations using a deep learning model, such as one implementing self-attention. In some examples, the one or more processors is configured to evaluate the loss function of the predicted performance associations among the plant genotypes and location-specific environment interactions with respect to their true grouping values. In some examples, the one or more processors is configured to adjust the weights of the deep learning model, e.g., a deep learning model implementing self-attention, and/or an embedding model, and/or a predictive output layer of tokens to reduce the evaluated loss. In some examples, the one or more processors is configured to reiterate and perform the operations of obtaining the at least one training data set; simultaneously learn from the training data set; evaluate the loss function of the predicted performance associations and adjust the weights of the the deep learning model, e.g., a deep learning model implementing self-attention, and/or an embedding model, and/or a predictive output layer of tokens until convergence of validation loss to a desired value. In some examples, the one or more processors is configured to receive as input into the trained deep learning model representations of genotypic data for two or more candidate plant genotypes and location-specific environmental data and spatial coordinate data as embedding vectors and generate a performance prediction for the phenotype of interest for one or more candidate plant genotypes as compared to a reference candidate plant genotype.

In some embodiments, the systems may include one or more servers and a computing device communicatively coupled to the one or more servers. One or more of the servers may include genotypic data, location-specific environmental data, and spatial coordinate data associated with two or more candidate plant genotypes. The computing device may include a memory and one or more processors configured to perform operations. In some examples, the one or more processors is configured to receive as input into a trained deep learning model representations of genotypic data for two or more candidate plant genotypes and location-specific environmental data and spatial coordinate data as embedding vectors and generate a performance prediction for a phenotype of interest for one or more candidate plant genotypes as compared to a reference candidate plant genotype.

### BRIEF DESCRIPTION OF THE DRAWINGS

FIG. 1. is a block diagram illustrating an exemplary computer system including a server and a computing device according to an embodiment as disclosed herein.

FIG. 2. is a schematic illustrating the input (training data), output (prediction of probability of relative performance of a training plant genotype for at least one phenotype as compared to another training plant genotype at a given location), and target (observation of which plant genotype outperformed the other) in one embodiment of training a machine learning model using supervised learning to learn genotype by environment interactions for a plurality of plants at various locations, for example, learn how phenotypes of the genotypes relate to one another in a given environment.

FIG. 3. is a schematic illustrating the input (training data), output (prediction of difference in performance of a training plant genotype for at least one phenotype as compared to another training plant genotype at a given location), and target (observation of the difference in at least one phenotype for a training plant genotype in comparison to another training plant genotype) in one embodiment of training a machine learning model using supervised learning to learn genotype by environment interactions for a plurality of plants at various locations, for example, learn how phenotypes of the genotypes relate to one another in a given environment.

FIG. 4. is a schematic illustrating the input (candidate genotype data for two different candidate plants and location-specific environmental data for a given location) and output (prediction of probability of relative performance of a candidate plant for at least one phenotype compared to another candidate plant at a given location) in one embodiment of a machine learning model established for learned genotype by environment interactions for a plurality of plants at various locations, e.g., where the model has learned how phenotypes of plant genotypes relate to one another in a given environment.

FIG. 5. is a schematic illustrating the input (candidate genotype data for two different candidate plants and location-specific environmental data for a given location) and output (prediction of difference in performance of candidate plant for at least one phenotype as compared to another candidate plant at a given location) in one embodiment of a machine learning model established for learned genotype by environment interactions for a plurality of plants at various locations, e.g., where the model has learned how phenotypes of plant genotypes relate to one another in a given environment.

FIG. 6. is a schematic illustrating the input (candidate genotype data for two different candidate plants and location-specific environmental data for a given location) and output (prediction of probability of relative performance of a candidate plant for at least one phenotype compared to another candidate plant at a given location) in one embodiment of a deep learning neutral network established for learned genotype by environment interactions for a plurality of plants at various locations.

FIG. 7. is a schematic illustrating the input (candidate genotype data for two different candidate plants and location-specific environmental data for a given location) and output (prediction of difference in performance of a candidate plant for at least one phenotype as compared to another candidate plant at a given location) in one embodiment of a deep learning neutral network established for learned genotype by environment interactions for a plurality of plants at various locations, e.g., where the model has learned how phenotypes of plant genotypes relate to one another in a given environment.

FIG. 8 is a schematic illustrating, in one embodiment, the ranking of a set of candidate plants based on the plants' predicted relative performance across a variety of genotype comparisons. For example, individual (candidate) plants may be ranked based on how many pairwise match-ups they are predicted to win (out-perform the other plant in the pair of plants) or lose (under-perform as to the other plant in the pair of plants) at one or more given locations as compared to the other plants in the set. For example, FIG. 8 is a schematic illustrating the input (data from a plurality of pairwise comparisons for a candidate plant's performance for at least one phenotype as compared with another plant in at least one or more given locations) and output (ranking of individual candidate plants based on probability of predicted performance for at least one phenotype at one or more given locations).

FIG. 9 is a schematic illustrating, in one embodiment, the ranking of a set of candidate plants based on the plants' predicted difference in performance. For example, individual (candidate) plants may be ranked based on their metric differences in phenotypic value at one or more given locations as compared to the other plants in the set. FIG. 9 is a schematic illustrating, in one embodiment, the input (data from a plurality of pairwise comparisons for a candidate plant's performance for at least one phenotype as compared with another plant in at least one or more given locations) and output (relative performance in the units of the phenotype of individual candidate plants based on difference in predicted performance for at least one phenotype at one or more given locations). FIG. 9 shows one embodiment of ranking of individual candidate plants from comparision events across or more specific locations.

FIG. 10A-10B shows the testing evaluation of one embodiment of a Deep Neural Network (DNN) genotype by environment (GxE) Model. DNN and Deep Learning Neural Network are used herein interchangeably. Validation approach 1 denotes evaluation of the model trained on all years 2012-2020 with a held-out set of hybrids. Validation approaches 2 and 3 correspond to the model trained with all hybrids from 2019 held out, evaluated on 2019 locations (2) and on 2012-2018 locations (3). The overall relationships between P (Hybrid 1 beats Hybrid 2) and the observed pairwise yield Best linear unbiased estimators (BLUE) differences is provided in (a). The blue histograms within (b) show the distributions of the difference between observed pair differences in locations where hybrid 1 was predicted to beat hybrid 2 vs. where hybrid 2 was predicted to beat hybrid 1, for pairs with at least 5 locations of each prediction in the observed set.

FIG. 11 shows one example of predictive GxE repeatability vs. GxE predictive accuracy. For validation approaches 2 and 3, predictions for hybrid pairs are divided into groups based on the correlation between the predictions of X as hybrid 1 and Y as hybrid 2, such that values closer to −1 indicate greater predictive consistency across locations. This is related to the difference of observed pair differences in locations where hybrid 1 was predicted to beat hybrid 2 vs. where hybrid 2 was predicted to beat hybrid 1, for pairs with at least 5 locations of each prediction in the observed set. For both validation approaches, the greater consistency of GxE predictions is associated with greater GxE prediction accuracy.

FIG. 12 shows the predictions of Hybrid A versus Hybrid B from Example 3. DNN predictions for Hybrid A, a high yielding hybrid under non-stressful conditions, and for Hybrid B, a drought-resistant hybrid are plotted for 2012—an extreme drought year—and 2018—a non-drought year (a). The relationship between the predictions and the true yield BLUE differences are shown below for the subset of locations with observed data for both hybrids (b).

FIG. 13 shows the predictions of new pre-commercial hybrids in an extreme drought year from Example 3. Visualizations of the predictions for a 2019 R2 (Hybrid C) and a 2020 R2 (Hybrid D) are provided against Hybrid A within the drought year of 2012 (a). Both show patterns of drought tolerance compared to Hybrid A, and this is supported with observed data for the 2019 R2 hybrid (b).

FIG. 14 shows one embodiment of multiple hybrid rankings in different environments. The 2020 R3 class is ranked, along with Hybrid A and Hybrid B in Western dryland 2012 environments. In the non-irrigated environments, the drought-resistant varieties—alongside several predicted drought-resistant hybrids—are predicted to have the highest yields on average. However, the varieties are predicted to switch rank ordering under irrigation.

FIG. 15 shows a schematic of one embodiment of encodings for two hybrids (Hybrid 1 and Hybird 2) and an environment (Environment A).

FIG. 16 shows an example of input of markers from a female parent and a male parent for a variational autoencoder (VAE) and output (for a decoder) for a hybird encoding in one embodiment.

FIG. 17 shows a schematic of one embodiment of an environmental encoding that includes an environment variational autoencoder (VAE) and an environment variational yield encoder.

FIG. 18 shows a flowchart for optimizing hyperparameters of genetic and environmental encoders, prior to tuning and training of a final predictor of GxE.

FIG. 19 shows an example of observed versus predicted pairwise yield differences for North America in 2019, where 2019 was completely held-out from training.

FIG. 20A shows a boxplot graph of within-pair yield differences grouped by a score of predictive consistency. Each datum within the boxplot represents the mean difference in yield for the predicted winner vs. predicted loser for pairs in which the first hybrid was predicted to beat hybrid 2 in at least 5 locations and the second hybrid was predicted to beat the first in at least 5 other locations.

FIG. 20B is a schematic showing how the two different genotypes may be input into the predictor using alternative configurations, allowing the derivation of a consistency score based on the agreement of predicted differences across locations.

FIG. 21A shows predicted versus observed yield differences for a held out set of locations.

FIG. 21B shows predicted versus observed differences of the product of yield and moisture across a held out set of locations.

FIG. 21C shows the predicted versus observed differences in adjusted gross income (AGI) across a held-out set of locations.

FIG. 22 is a schematic illustrating a field with multiple subfields. Each subfield has multiple plots, and each plot comprises a candidate plant genotype. G is an abbreviation for a candidate plant genotype. E is an abbreviation for location-specific environmental conditions within each subfield. S is an abbreviation for a spatial location of a subfield. In another embodiment, different candidate plant genotypes (e.g., G1, G2, G3, and G4) are predicted for performance for a phenotype of interest at individual plots within different subfield locations (e.g., S1, S2, S3) in a field, each of which may have its own environment conditions. In this embodiment, different candidate plant genotypes (e.g., G1, G2, G3, and G4) are predicted for relative performance for a phenotype of interest within a subfield (S1 or S2 or S3) having the same location-specific environmental conditions. Using the methods and systems described herein, one embodiment includes predicting the performance of a given spatial unit of a candidate plant genoytpe, e.g., G1,S1, E1, for a particular phenotype of interest relative to the predicted performance of a selected reference spatial unit for a different plant genotype, e.g., G2,S1,E1, for the particular phenotype to generate a predicted relative performance value, such as a probability that of one of the at least two candidate plant genotypes, e.g., G1,S1, E1, outperforms another candidate plant genotype, e.g., G2,S1,E1, for at least one phenotype at a given location, In another example, using the methods and systems described herein, one embodiment includes predicting the performance of a given spatial unit of a candidate plant genoytpe, e.g., G1,S2, E2, for a particular phenotype of interest relative to the predicted performance of a selected reference spatial unit for a different plant genotype, e.g., G2,S2,E2, for the particular phenotype to generate a predicted relative performance value, such as a probability that one of the at least two candidate plant genotypes, e.g., G1,S2, E2, outperforms another candidate plant genotype, e.g., G2,S2,E2, for at least one phenotype at a given location. Using the methods and systems described herein, one embodiment includes predicting the relative performance of two candidate plant genotypes in a pairwise comparison.

FIG. 23 is a schematic illustrating a field with multiple plots, where each plot comprises a candidate plant genotype within a spatial location, also called a subfield. G is an abbreviation for a candidate plant genotype. E is an abbreviation for location-specific environmental conditions encompassing the full field. S is an abbreviation for subfield location. In this embodiment, the subfields S1, S2, and S3 have a single environment (E1) with the same location-specific environmental conditions, for example, same soil type, temperature, precipitation, and irrigation. Each of the candidate plant genotypes (e.g., G1, G2, G3, and G4) are on different individual plots within each of the three subfields, S1, S2, and S3. The different candidate plant genotypes (e.g., G1, G2, G3, and G4) are predicted for performance for a phenotype of interest in an aggregation of plots of a given genotype across different locations (e.g., subfields S1, S2, S3) in a field, under the same environmental conditions (E1). The mean or Best linear unbiased estimators (BLUE) of each genotype on two or three plots may be determined. In some examples, the predicted performance of a plant genoytpe for a particular phenotype of interest may be averaged between or across plots on two or more or all locations, e.g., S1, S2, and S3; or S1 and S2; or S1 and S3; or S2 and S3, under the same environmental conditions. The average predicted performance of the plant genotype, e.g., G1, for the particular phenotype, e.g., yield, for a given spatial unit, e.g., plot, may then be compared to an average predicted performance of a selected reference spatial unit for a different plant genotype, e.g., G2, for the particular phenotype to generate a predicted relative performance value, such as a probability that one candidate genotype, e.g., G1, outperforms another candidate plant genotype, e.g., G2, for at least one phenotype at a given location. The predicted performance of the candidate plant genoytpe, e.g., G1, for a particular phenotype of interest, e.g. yield, based on the aggregate across subfields (e.g. S1, S2, S3) may be compared with the aggregate performance of a selected reference genotype, e.g., G3, within the same field to generate a predicted relative performance value, such as a probability that the candidate plant genotype, e.g., G1, outperforms the reference candidate plant genotype, e.g., G3.

FIG. 24 is a schematic illustrating a field with multiple subfields. Each subfield has multiple plots with each plot having a candidate plant genotype. G is an abbreviation for a candidate plant genotype. E is an abbreviation for location-specific environmental conditions within each subfield. S is an abbreviation for spatial location of a subfield. In this embodiment, different plant genotypes (G1, G2, G3, and G4) are predicted for performance for a phenotype of interest for individual plots within several subfields, e.g., S1, S2, and S3, each having varying location-specific environmental conditions, e.g., E1, E2, and E3. In one embodiment, the methods and systems described herein use a transformer deep learning model to predict performance of one or more candidate plant genotypes for a particular phenotype of interest on a particular plot, e.g., G4, S1, E1, and G3, S2, E2, as compared with the predicted performance of a reference plot of a different and/or same candidate plant genotype, e.g. G4, S3, E3, for the phenotype of interest within the same and/or different spatial locations, e.g., subfields, e.g., S1, S2, or S3, and within the same and/or different location-specific environmental conditions (E1, E2, or E3) to generate predicted relative performance values, e.g. differences.

FIG. 25 is a schematic illustrating a field with multiple subfields. Each subfield has multiple plots, with each plot having a candidate plant genotype. G is an abbreviation for a candidate plant genotype. E is an abbreviation for location-specific environmental conditions within a subfield. S is an abbreviation for spatial location of a subfield. In this embodiment, different plant genotypes (G1, G2, G3, and G4) are predicted for performance for a phenotype of interest for individual plots within several subfields, e.g., S1, S2, and S3, under different environmental conditions (E1, E2, E3). In one embodiment, the methods and systems described herein use a deep learning transformer model to predict performance of one or more candidate plant genotypes for a particular phenotype of interest on a particular plot as compared with the predicted performance of a reference spatial unit of different and/or same candidate plant genotypes for the phenotype of interest on one or more particular plots within the same and/or different locations, e.g., subfields, e.g., S1, and/or different environmental conditions to generate predicted relative performance values.

FIG. 26 is a schematic illustrating the input (training data), output (prediction of value, difference, or probability of relative performance of one or more training plant genotypes for at least one phenotype as compared to another plant genotype, e.g., a reference plant genotype, each at given locations in the field), and target (observation comparing training plant genotypes' measured performances for a phenotype of interest versus reference plant genotype, each at their respective locations in the field) in one embodiment of training a deep learning model, e.g., a deep learning transformer model using self-attention, to learn the relationship of the genotype by environment interactions with the phenotypic performance for a plurality of genotypes at various subfield locations. G is an abbreviation for a candidate plant genotype. S is an abbreviation for a subfield.

FIG. 27 is a schematic illustrating the input (input data) into a trained deep learning transformer model, and output (prediction of value, difference, or probability of relative performance of one or more of the candidate plant genotypes for at least one phenotype as compared to another plant genotype, e.g., a reference plant genotype, at a given location) in one embodiment of using a trained deep learning model, e.g., a deep learning transformer model using self-attention, that has been trained to learn genotype by environment interactions, e.g., the relationship of genotype by environment interactions with the phenotypic performance for a plurality of plant genotypes.

### DETAILED DESCRIPTION

It is to be understood that this invention is not limited to particular embodiments, which can, of course, vary. It is also to be understood that the terminology used herein is for the purpose of describing particular embodiments only, and is not intended to be limiting. Further, all publications referred to herein are each incorporated by reference for the purpose cited to the same extent as if each was specifically and individually indicated to be incorporated by reference herein.

Every year, breeders evaluate lines and make decisions regarding what lines should be selected, crossed, and advanced to create a product or variety in their plant breeding pipeline that has certain desirable traits or properties for a particular market or geography. In addition to the plant's genetics, breeders also take into consideration the plant's expected performance across multiple locations and environmental conditions when making selection and advancement decisions for lines and products. This is because a genotype can perform differently in diverse environments due to genotype by environment interactions. Genotype by environment interactions with respect to a plant refers to a change in the relative performance of at least two plant genotypes for a particular phenotype, as observed in at least two environments. To better understand which genotypes are expected to perform well in certain environments but not others, which genotypes are adaptable and stable across a range of environments, most breeding programs conduct multi-environmental field trial testing spanning years. However, multi-environment experiments are costly and labor-intensive processes.

In some embodiments, the methods and systems described herein enable the machine learning of genotype by environment interactions for plant performance at a given location using plant genotypic data and location-specific environmental data. The trained machine learning model may be used in any number of applications, for example, in systems and methods of predicting performance of candidate plant genotypes for at least one phenotype of interest at one or more given locations, including single or aggregated locations. Candidate plant genotypes may include those plant genotypes being considered for any number of applications, e.g. candidate plant genotypes for selection, candidate plant genotypes for planting in a region, candidate plant genotypes for crossing, and/or other applications in a breeding pipeline. In some examples, the candidate plant genotypes may include plants used in training the machine learning model, validating the machine learning model, or plants that the established machine learning model has not seen before (new plant genotypes), or combinations thereof. Candidate plants may include but are not limited to plants from different families, plants from different breeding programs, or plants from different stages in the pipeline.

In some embodiments, the systems and methods include predicting for at least one or more spatial units of candidate plant genotypes a performance for a phenotype of interest. In some examples, the spatial unit is a pot, a row, a plot, a subfield, or a field. In some embodiments, the predicted performance is a relative performance. For example, the performance of one or more spatial units of candidate plant genotypes for a phenotype of interest is compared against a predicted performance for the phenotype of interest for a reference spatial unit of one or more plant genotypes at a certain location. A spatial unit may be a pot, a row, a plot, a subfield, a field, or any other unit of space useful for growing one or more plants. A reference spatial unit is a spatial unit with a specific plant genotype at a certain location that is selected for performance comparison purposes with the one or more training plant genotypes or candidate plant genotypes. The reference spatial unit and the other training or candidate (non-reference) spatial units are of the same unit, for example, if the spatial unit for a non-reference plant genotype is a plot, then the reference spatial unit will also be a plot. As a non-limiting example, when the phenotype of interest is yield, the prediction difference between the yields of candidate plant genotype A and candidate plant genotype B may be expressed in units of bushels/acre for the genotype for a particular location, e.g. a particular plot.

In some embodiments, the systems and methods include predicting the performance of at least two or more candidate plant genotypes, for a given spatial unit, e.g., a plot, for at least one phenotype at one or more given locations using a trained machine learning model, for example, a deep learning model or a supervised learning model. In some examples, the prediction is a probability of one of the at least two candidate plant genotypes outperforming another candidate plant genotype for at least one phenotype at one or more given locations.

In some examples, the prediction is the difference between or among the predicted performance of one or more of the candidate plant genotypes for at least one phenotype of interest as compared to the predicted performance for a reference candidate plant genotype for the same phenotype at a given location and spatial unit. In some examples, the trained machine learning model predicts whether one or more of the candidate plant genotypes that are not the reference candidate plant genotype will perform better for the phenotype of interest compared to the reference candidate plant genotype.

In some examples, the prediction is the difference between the performance of a candidate plant genotype for at least one phenotype as compared to another plant genotype's performance for the same phenotype at a given location. For example, the trained machine learning framework may predict whether one hybrid genotype will out-yield another hybrid genotype within a given location based on their genotypes and the environmental conditions. In some examples, the machine learning framework may incorporate representations of spatial coordinate data in addition to the plant genotype data and location-specific environmental data to make predictions at the plot level. In some examples, spatial coordinate data includes the relative coordinates or positioning of one or more rows or plots within a subfield or field, for example, the row, column coordinates of a plot within a subfield or a field. In some aspects, the spatial coordinate data does not include environmental spatial information such as temperature, precipitation, soil conditions, and management.

In some examples, the prediction is the difference between the average performance of a candidate plant genotype for at least one phenotype as compared to another plant genotype's average performance for the same phenotype within a spatial unit, e.g., row, plot, subfield, or field. If desired, the performance predictions can be compared across multiple plant genotypes and locations to rank individual plants across one or multiple locations or environments of interest. In some examples, if the comparisons are pairwise, the performance predictions may be compared across multiple pairs and locations to rank individual plants across one or multiple locations or environments of interest if desired.

The modeled (learned) genotype by environment interactions may used to make predictions about performance for new (unobserved) environments and/or new (unobserved) candidate plant genotypes, or combinations thereof for candidate plant genotypes and/or locations. In one embodiment, use of the learned genotype by environment interactions enables the comparison of existing and/or potential products' performances in environments with known environmental conditions, including stress.

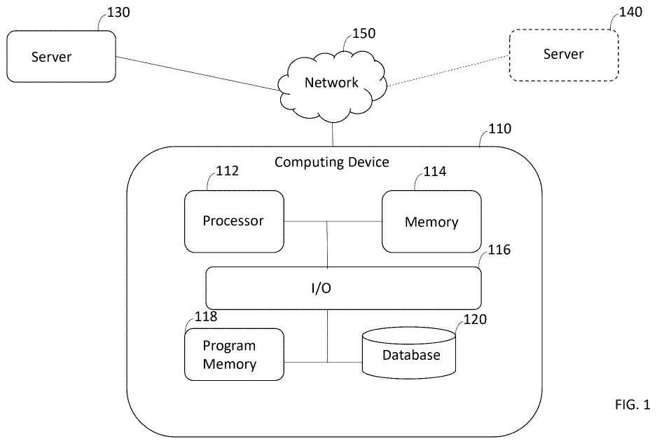

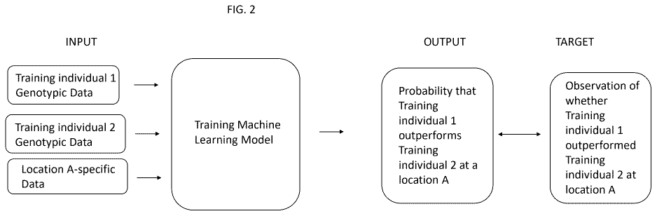

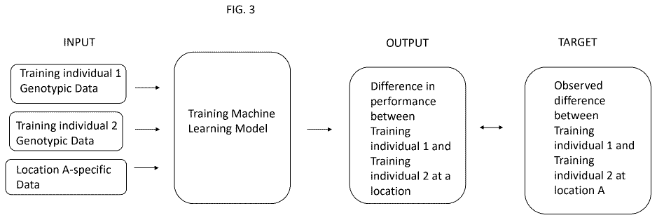

Referring to FIG. 1, a block diagram of a computer system 100 for learning the relationship of genotype by environment interactions with the phenotypic performance for a plurality of plant genotypes and creating predictions for plant genotype performance is shown. To do so, the system 100 may include a computing device 110 and a server 130 that is associated with a computer system. The system 100 may further include one or more servers 140 that are associated with other computer systems such that the computing device 110 may communicate with different computer systems running different platforms. However, it should be appreciated that, in some embodiments, a single server (e.g., a server 130) may run multiple platforms. The computing device 110 is communicatively coupled to the one or more servers 130, 140 via a network 150 (e.g., a local area network (LAN), a wide area network (WAN), a personal area network (PAN), the Internet, etc.).

In an embodiment, the computing device 110 may generate predictions of plant genotype performance by using at least one machine learning model to generate a predicted probability or difference for the relative performance for at least one or more candidate plant genotypes for at least one phenotype of interest versus a reference candidate plant genotype at one or more locations, for example, the predicted probability that one hybrid genotype out-yields another hybrid genotype at a single location.

In an embodiment, the computing device 110 may generate predictions of plant genotype performance by using at least one machine learning model to generate a predicted probability for the relative performance for at least one candidate plant genotype pair for at least one phenotype of interest, for example, the predicted probability that one hybrid genotype out-yields another hybrid genotype at a single location.

In another embodiment, the computing device 110 may generate predictions of relative plant performance by using at least one machine learning model to generate a predicted difference in performance between at least one candidate plant genotype pair for at least one phenotype of interest: for example, the predicted difference between the yields of hybrid genotype A and hybrid genotype B in units of bushels/acre at a single location.

More specifically, the computing device 110 may obtain data stored in a database 120, input, and/or downloaded by a user. For example, in the context of predicting one or more candidate plant genotypes' performances, a machine learning model may be trained to learn genotype by environment interactions for multiple different plant genotypes and locations, e.g. from one or more training datasets. For example, the machine learning model may be trained to learn the relationship of genotype by environment interactions with the phenotypic performance for a plurality of plant genotypes, e.g., learn how phenotypes of the genotypes relate to one another in a given environment. In some embodiments, the trained machine learning model uses the learned genotype by environment interactions to a predict the probability of one or more candidate plant genotype's performance for at least one phenotype of interest as compared to another candidate plant genotype's performance for the same phenotype(s) at one or more given locations. In some embodiments, the trained machine learning model uses the learned genotype by environment interactions to predict the difference in performance between at least one or more candidate plant genotypes for at least one phenotype at one or more given locations as compared to a reference plant genotype, e.g., a reference candidate plant genotype. In some examples, the trained machine learning model uses the learned genotype by environment interactions to predict the difference in performance between at least one candidate plant pair for at least one phenotype at a given location.

The one or more training datasets may include but are not limited to data representations of plant genotypes and location-specific environmental data. In some aspects, the genotypic data includes information about the genome of a given plant or plants, for example, a collection of genotypic markers, such as genome-wide markers, a specific subset of genotypic markers, presence or absence in the genome of specific mutations, single nucleotide polymorphisms (SNPs), insertion of bases, deletion of bases, other sequence information, or any combination thereof. For example, genotypic data may include genome-wide marker information, genome sequence information selected from the group consisting of SNP, QTL, RNA-seq, short read genomic sequencing, marker data, long read genome sequence information, methylation status, gene expression values, indels, haplotypes, and combinations thereof.

In some examples, the data for genomic information of the plants may be obtained using high density DNA arrays, PCR-based methods, including tape arrays, TaqMan assays, Restriction Fragment Length Polymorphisms (RFLPs), Target Region Amplification Polymorphisms (TRAPs), Isozyme Electrophoresis, Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), or any combinations thereof.

In some examples, the genotypes, phenotypes, or specific-location environmental data include information that is imputed, inferred, or predicted rather than directly measured.

In some examples, the genotypic information, such as marker information, is imputed. In some examples, the marker information is imputed based on a common latent representation of the underlying marker information using global and/or local variational autoencoders (VAEs). See, for example, Example 4, and U.S. Pat. No. 11,174,522, granted Nov. 16, 2021, hereby incorporated by reference in its entirety.

In some aspects, the location-specific environmental data includes but is not limited to information for or relating to geographical locations such as latitude and longitude information, land features such as elevation, site topography, climate conditions e.g. weather conditions, including but not limited to wind direction, wind velocity, cloud cover, humidity, relative humidity, sunrise, sunset, temperature, precipitation, water vapor, vapor pressure deficit, snow depth, barometric pressure, season, heat index, visibility, dew point, air quality, storms, solar radiation. In some aspects, environmental data may be obtained from remotely-sensed imagery, including visual light, infrared, near-infrared, multi-spectral, and hyperspectral imaging bands, in some aspects, these bands may be combined into vegetative indices, including but not limited to the normalized difference vegetation index (NDVI), enhanced vegetation index (EVI), weighted difference vegetation index (WDVI), or normalized difference water index (NDWI); soil type or soil substrate type e.g. sand, loam, clay, soil conditions e.g. aeration level, temperature, (ground) water level, soil moisture, humidity level, pH, composition such as organic matter, degree of compaction, ground soil organic carbon estimates or capacity, soil toxicities, soil nutrients, inputs or applied products such as fertilizers, herbicides, insecticides, seed treatments, seed- or soil-applied agricultural biologicals soil drainage; (crop) plant conditions e.g. plant population density, planting date, nutrient application, plant height, evapotranspiration rate, gross primary productivity (GPP), growing and harvesting season, growth cycle, stage of development, such as plant phenological stage-including flowering and grain filling, green-up, dry down, and senescence, seed type, vegetation, crop variety, chemical, physical and nutritional requirements; biotic stresses such as plant disease resistance level, including but not limited to Northern Leaf Blight (NLB) and Goss's Wilt (GOSWLT, plant herbicide tolerance level, physical injuries e.g. from pathogens, herbicides, storms, plant stress; abiotic stresses such as early and late root lodging, stalk lodging, brittlesnap, willowing, management decisions such as row count, irrigation, irrigation location, and tillage; previous and/or subsequent crops in a crop rotation system, crop production methods, e.g. crops grown in the open field, in a growth chamber, or in a greenhouse; disease and pest events, weeds, or any combinations thereof.

The location-specific environmental data may be obtained in any suitable manner or using any technique, including without limitation imaging devices, cameras, and sensors. The data may be from any number of sources, including but not limited to an aerial source, including but not limited to one or more satellites, including high resolution satellites, airplanes, helicopters, balloons and UAV platforms; a ground-based source including but not limited to one or more trucks, tractors, rovers, or other vehicles or objects such as weather stations that are land-bound, or a mobile-source including but not limited to one or more hand-held or mobile devices, or sources of data that do not fall into any of the other categories.

The training plant genotype or data may be from a plant at any stage of development or growth, including an immature plant or mature plant, including a plant at harvest time.

The data used in the methods and systems herein may be obtained from one or more sources such as an external database, an internal database, a private data source, a public data source, or combinations thereof.

Non-limiting examples include public weather information from public databases and sources such as the National Oceanic and Atmospheric (NOAA), National Weather Service (NWS), Meteorological Simulation Data Ingest System (MADIS), Esri Open Data Portal, National Aeronautics and Space Administration (NASA), European Space Agency, Natural Resources Conservation Service (NRCS-SSURGO), United States Geological Survey (USGS), and Parameter-elevation Regressions on Independent Slopes Model (PRISM); public soil information from public databases and sources such as the United States Department of Agriculture (USDA) SSURGO soil survey database.

Accordingly, the methods and systems described herein may use collected, stored, or retrieved data, or combinations thereof. The data, including genotypic, location-specific environmental, and spatial, may be real time information, historical information, and/or predicted information.

In some examples, the data comes from plants grown in a pot, a row, a plot, a subfield, a field, or a greenhouse. In some examples, the data may be obtained from any suitable plants or parts thereof, for example, cells, seeds, leaves or plants at any various plant growth and developmental stages, such as immature plants, seedlings, and mature plants including at harvesting.

In some examples, the plant genotypes are inbred plant genotypes, hybrid plant genotypes, varietal genotypes, genotypes from immediate and subsequent generations, offspring genotypes or progeny genotypes thereof, or any combination of one or more of the foregoing. In some examples, the plants are inbred plants, hybrid plants, varieties, immediate and subsequent generations, offspring or progeny thereof, or any combination of one or more of the foregoing. Any monocot or dicot plant may used with the methods and systems provided herein, including but not limited to a soybean, maize, sorghum, cotton, canola, sunflower, rice, wheat, sugarcane, alfalfa tobacco, barley, cassava, peanuts, millet, oil palm, potatoes, rye, or sugar beet plant.

The machine learning model may be trained to learn the the relationship of genotype by environment interactions with the phenotypic performance for a plurality of plant genotypes from one or more training datasets so the trained model is capable of predicting performance for a phenotype of interest for one or more candidate plant genotypes, e.g., whether the one or more candidate plant genotypes will perform better or worse for at least one phenotype of interest at one or more given locations when compared to one or more reference plant genotypes.

In one embodiment, the machine learning model may be trained to learn genotype by environment interactions from one or more training datasets so the trained model is capable of predicting which training plant in a pair of training plants will perform better or worse for at least one phenotype at a given location when compared to the training plant in the pair of training plants.

In one embodiment, the machine learning model may be trained to learn the relationship of genotype by environment interactions with the phenotypic performance for a plurality of plant genotypes from one or more training datasets so the trained model is capable of predicting which training plant in a pair of training plants will perform better or worse for at least one phenotype at a given location when compared to the training plant in the pair of training plants.

In some examples, the one or more training datasets may be selected based on plant genotypes of interest, plant phenotypes of interest, genetic values obtained from MLE, environmental conditions of interest, geographic regions, management zones, maturity ranges, and/or additional considerations or combinations thereof. In some examples, the training datasets may be further selected based on additional considerations, for example, specific years, and the needs for evaluation of trained predictors in hold-out sets. In some embodiments, the one or more training datasets may be selected based on training data that has the phenotypes of interest on genotyped material with locations and years that have environmental data available.

In general, the computing device 110 may include any existing or future devices capable of training a machine learning model or using the trained machine learning model. For example, the computing device may be, but is not limited to, a computer, a notebook, a laptop, a mobile device, a smartphone, a tablet, wearable, smart glasses, or any other suitable computing device that is capable of communicating with the server 130.

The computing device 110 includes a processor 112, a memory 114, an input/output (I/O) controller 116 (e.g., a network transceiver), a memory unit 118, and a database 120, all of which may be interconnected via one or more address/data bus. It should be appreciated that although only one processor 112 is shown, the computing device 110 may include multiple processors. Although the I/O controller 116 is shown as a single block, it should be appreciated that the I/O controller 116 may include a number of different types of I/O components (e.g., a display, a user interface (e.g., a display screen, a touchscreen, a keyboard), a speaker, and a microphone).

The processor 112 as disclosed herein may be any electronic device that is capable of processing data, for example a central processing unit (CPU), a graphics processing unit (GPU), a system on a chip (SoC), or any other suitable type of processor. It should be appreciated that the various operations of example methods described herein (i.e., performed by the computing device 110) may be performed by one or more processors 112. The memory 114 may be a random-access memory (RAM), read-only memory (ROM), a flash memory, or any other suitable type of memory that enables storage of data such as instruction codes that the processor 112 needs to access in order to implement any method as disclosed herein. It should be appreciated that, in some embodiments, the computing device 110 may be a computing device or a plurality of computing devices with distributed processing.

As used herein, the term “database” may refer to a single database or other structured data storage, or to a collection of two or more different databases or structured data storage components. In the illustrative embodiment, the database 120 is part of the computing device 110. In some embodiments, the computing device 110 may access the database 120 via a network such as network 150. The database 120 may store data (e.g., input, output, intermediary data) used for predicting plant performance. For example, the data may include genotypic data, phenotypic data, location-specific environmental data, mean locus effects (MLE) data, predicted genetic values, pedigree information, co-ancestry information, or combinations thereof that are obtained from one or more servers 130, 140.

The computing device 110 may further include a number of software applications stored in a memory unit 118, which may be called a program memory. The various software applications on the computing device 110 may include specific programs, routines, or scripts for performing processing functions associated with the methods described herein. Additionally or alternatively, the various software applications on the computing device 110 may include general-purpose software applications for data processing, database management, data analysis, network communication, web server operation, or other functions described herein or typically performed by a server. The various software applications may be executed on the same computer processor or on different computer processors. Additionally, or alternatively, the software applications may interact with various hardware modules that may be installed within or connected to the computing device 110. Such modules may implement part of or all of the various exemplary method functions discussed herein or other related embodiments.

Although only one computing device 110 is shown in FIG. 1, the server 130, 140 is capable of communicating with multiple computing devices similar to the computing device 110. Although not shown in FIG. 1, similar to the computing device 110, the server 130, 140 also includes a processor (e.g., a microprocessor, a microcontroller), a memory, and an input/output (I/O) controller (e.g., a network transceiver). The server 130, 140 may be a single server or a plurality of servers with distributed processing. The server 130, 140 may receive data from and/or transmit data to the computing device 110.

The network 150 is any suitable type of computer network that functionally couples at least one computing device 110 with the server 130, 140. The network 150 may include a proprietary network, a secure public internet, a virtual private network and/or one or more other types of networks, such as dedicated access lines, plain ordinary telephone lines, satellite links, cellular data networks, or combinations thereof. In embodiments where the network 150 comprises the Internet, data communications may take place over the network 150 via an Internet communication protocol.

Described herein are methods and systems for predicting plant performance for at least one phenotype of intertest that include using an established, i.e. trained, machine learning model. In some examples, the machine learning model is a deep learning model or a supervised learning model. In some examples, the deep learning model is a deep learning model implementing self-attention. In some examples, the machine learning model is a deep learning transformer model. In some examples, the machine learning model is a deep learning model implementing self-attention. The machine learning model may be established by using, as input for training, data representations of genotypes and location-specific environmental information, with phenotypes (phenotypic performance data) used to derive target outputs. In some examples, the machine learning model may additionally use data representations for spatial coordinates for the spatial uint, e.g. a row, a plot, a subfield, or a field.

In some examples, one or more training datasets may be selected as input for the machine learning model based on the user preference, environmental conditions, geographic regions, collection years, plant genotypes, plant phenotypes, genetic values obtained from MLE, requirements for held-out data evaluation of the trained predictor, and/or additional considerations, or combinations thereof. While the data can be confined to one particular year or period of time of interest if desired, in some examples, the data in the training dataset is from multiple years, e.g. from at least 2, 3, 4, 5, 6, 7, 8, 9, 10, 11, 12, 13, 14, 15, 16, 17, 18, 19, 20, or more years.

One or more machine learning models may be trained to predict a plant genotype's performance for at least one phenotype of interest at one or more given locations. Data from one or more training datasets comprising plant genotypes and location-specific environment information may be used to train the machine learning model to learn how phenotypes of the plant genotypes relate to one another in a given environment, e.g., the relationship of genotype by environment interactions with the phenotypic performance for a plurality of plant genotypes. The trained machine learning model may be used in the methods and system described herein. In some embodiments, data from one or more training datasets comprising plant genotypes, location-specific environment information, and spatial coordinates may be used to train a deep learning model to learn the relationship how phenotypes of the genotypes relate to one another in given environment interactions. The trained deep learning model may be used in the methods and system described herein.

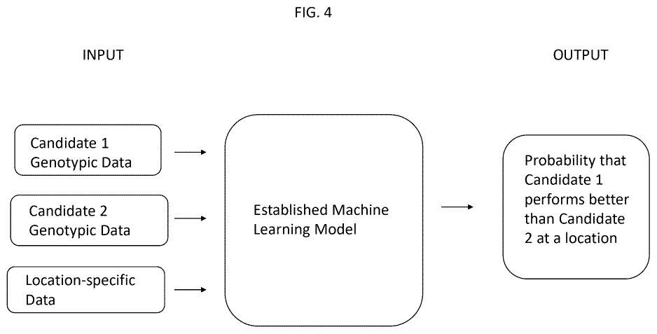

Referring now to FIG. 4, FIG. 4 is a schematic diagram illustrating one embodiment of using a trained machine learning model to generate a predicted probability for the relative performance for at least one candidate plant pair for at least one phenotype at a given location, for example, the predicted probability that hybrid A out-yields hybrid B at a single location.

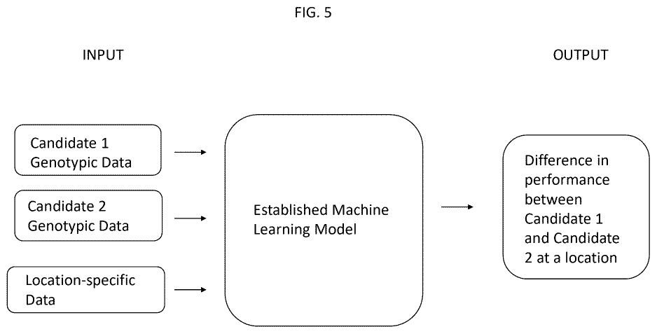

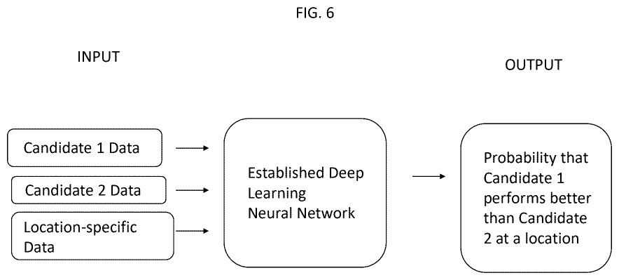

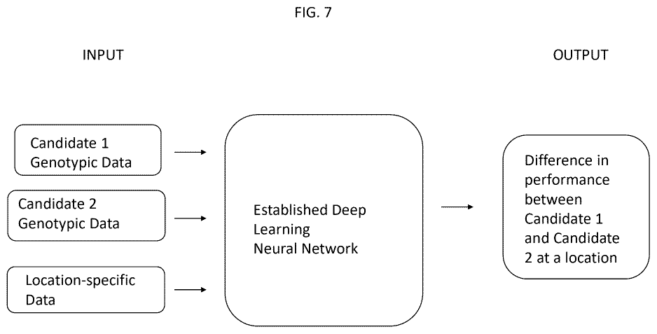

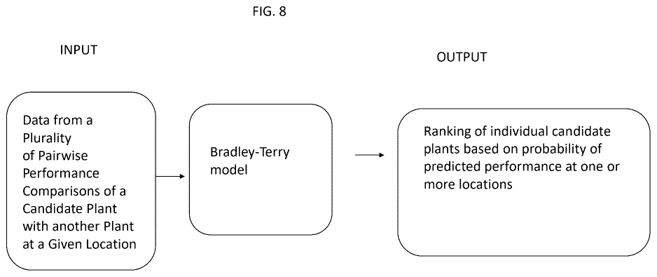

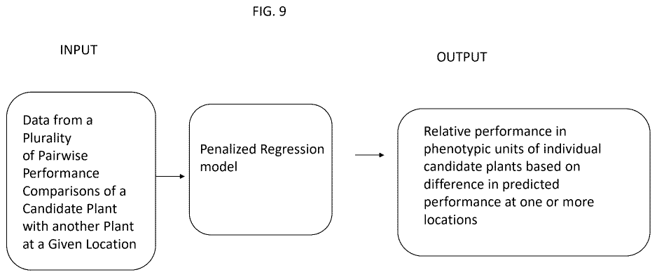

Referring now to FIG. 5, FIG. 5 is a schematic diagram illustrating one embodiment where the trained machine learning model uses the learned genotype by environment interactions to predict the difference in performance between at least one candidate plant pair for at least one phenotype at a given location.

The one or more machine learning models may be trained to learn genotype by environment interactions between genotype and location-specific environmental information and phenotype performance. In some embodiments, the one or more machine learning models may be trained to learn the relationship of genotype by environment interactions with phenotype performance using genotype, location-specific environmental, and spatial coordinate information. The model architectures, model weights, and any pre- or post-processing factors, may be written and stored so the models may be used to predict performance of candidate plants with new (outside training data) genotypes in new (outside training data) environments, candidate plants with genotypes in the training data in new (outside training data) environments, or candidate plants with new (outside training data) genotypes in environments observed in the training data.

Any suitable machine learning models may be used in the methods and systems described herein. Types of models include, without limitation, statistical models, machine learning models, and models involving deep learning, including fully-supervised, self-supervised, or semi-supervised methodologies. In some aspects, the machine learning model uses attention or self-attention mechanisms. In some aspects, the machine learning model is a classification model, a regression model, a distribution model, for example, a multivariate or univariate Gaussian distribution model, or a deep learning model, such as a transformer model. In some embodiments, the machine learning model is part of an ensemble model.

In some aspects, the machine learning model is a deep learning model, such as a deep learning transformer model. In some embodiments, the deep learning model is a supervised learning model. The supervised learning model may be a classification or regression model. The machine learning models include but are not limited to support vector machines, shallow neural networks, deep neural networks, ensembles of decision trees such as random forests or boosted tree ensembles, generalized additive models, Gaussian Processes, or generalizations of reproducing kernel Hilbert spaces (RKHS).

Described herein are methods and systems for predicting performances for a phenotype interest for one or more candidate plant genotypes that includes using an established deep learning model. In one embodiment, the deep learning model may be established by using, as input data, representations of genotypes from plants that have different known genotypes and location-specific environmental conditions and using as target outputs measured phenotypes for at least one phenotype or trait of interest within a common location and environment. In some aspects, the deep learning transformer model is established using data representations obtained from two or more genotypes grown under the different environments and locations.

The deep learning transformer model may be established by using, as input data, representations of genotypes from plants that have different known genotypes, location-specific environmental conditions, and spatial coordinates and using as target outputs measured phenotypes for at least one phenotype or trait of interest within a common location and environment. In some aspects, the deep learning transformer is established using data representations obtained from two or more genotypes grown under multiple environments and locations.

In some embodiments, the deep learning models may use any appropriate model in the methods and systems described herein, including any model that is capable of receiving as input the genotype-specific representations of at least two plant genotypes and the spatial unit representations, e.g., field or subfield-specific representations, of at least one environment known or assumed to contain the genotypes. The model may be constructed in any suitable way so that the predicted relative performance of the genotype is capable of varying among environments in magnitude, in sign, or both. In some embodiments, the model may include specific deep learning architectures such as dense layers, a convolutional layer, transformer layers (transformer model), recurrent layers, residual connections, or invertible transformations.

In some embodiments, the deep learning models, e.g., a deep learning transformer model, may use any appropriate model that does not a priori represent any consistent distinction among known genotypes, phenotypes, and the environments of the two or more plant genotype pairs prior to statistical analysis.

Described herein are methods and systems for predicting relative performances for a phenotype interest for a pair of candidate plant genotypes that includes using an established supervised learning model. The supervised learning model may be established by using as input data representations from pairs of plants that have different known genotypes and measured phenotypes for at least one trait of interest within a common location having environmental data for which a representation is used as a third input. In some aspects, the supervised learning model is established using data representations obtained from two or more genotypes grown under multiple environments and locations.

In some embodiments, the supervised learning models may use any appropriate model that does not a priori represent any consistent distinction among known genotypes, phenotypes, and the environments of the two or more genotype pairs or genotypes prior to statistical analysis. In some embodiments, the supervised learning model is a classification model. In some embodiments, the supervised learning model is a regression model. In some aspects, the supervised learning model uses multivariate analysis of the data representations, e.g. from datasets of the two or more genotypes, to relate the one or more data representations of plant genotypes and the environments to phenotypes.

Some non-limiting examples of machine learning algorithms that can be used to generate and update the engineered predictor features, post-prediction processors, or pairwise prediction models can include supervised and non-supervised machine learning algorithms, including regression algorithms (such as, for example, Ordinary Least Squares Regression or Ridge Regression), instance-based algorithms (such as, for example, Learning Vector Quantization), decision tree algorithms (such as, for example, classification and regression trees), Bayesian algorithms (such as, for example, Naive Bayes and Bayesian Neural Networks), clustering algorithms (such as, for example, k-means clustering), association rule learning algorithms (such as, for example, Apriori algorithms), artificial neural network algorithms (such as, for example, Perceptron), deep learning algorithms (such as, for example, Convolutional Neural Networks, Residual Neural Networks, or transformer-based models), dimensionality reduction algorithms (such as, for example, Principal Component Analysis), ensemble algorithms (such as, for example, Random Forests or Gradient-Boosted Trees), and/or other machine learning algorithms. In some embodiments, the supervised learning model is a deep learning neural network.

Further embodiments of the methods include establishing a supervised learning model using support vector machines or neural networks and the data representations as input. As used herein, “support vector machines” describe statistical analyses that establish a boundary between class members based on maximizing training example distances (using a kernel-derived metric) from a separating hyperplane. The term “neural network” is intended to mean—without restriction—the general composition of two or more mathematical functions, wherein each subfunction, or layer, is defined by one or more initial affine transformations of a tensor, followed by a nonlinear transformation through an “activation function”.

In some aspects, pre-processing steps are used to reduce the noise and dimensionality of the data prior to establishing the machine learning models predictive of relative performance, e.g. deep learning models. In some aspects, the pre-processing steps reduce the noise and dimensionality of large data sets. As used herein, “pre-processing” of the data sets means to apply statistical analyses to the raw data in order to reduce the noise and dimensionality of the data. The term “dimensionality” refers to the number of variables under consideration in a data set. The term “noise” refers to the presence of any signal in the data set other than the signals which are desired for analysis. One or more statistical analyses or mathematical pretreatments may be used to pre-process the data to reduce the noise and dimensionality, including but not limited to quantization, normalization, autoencoder embedding, multiplicative scatter correction, autoscaling, derivatization, or combinations thereof.

In some embodiments, the methods and systems include establishing a deep learning model, such as a deep learning transformer model, that can use a data representation of one or more candidate plant genotypes to predict the relative performance for at least one phenotype of interest as compared to a reference plant genotype, e.g., a reference candidate plant genotype, for one or more given locations and/or environmental conditions. In some aspects, the data representations of the candidate plant genotypes, location-specific environments, relative spatial coordinate data, and reference designation is used as input into the established deep learning transformer model.

In some examples, methods for predicting the performance for at least one phenotype of interest for at least one or more candidate plant genotypes includes inputting the data representations from the one or more candidate plant genotypes, location-specific environments, spatial coordinates, and designed reference into the established deep learning transformer model. In some examples, the deep learning transformer model may be trained to learn the relationship of genotype by environment interactions with the phenotypic performance for a plurality of plant genotypes (genotype by environment interactions). In some aspects, the deep learning transformer model is trained to learn to predict performance for at least one phenotype of interest for at least one or more candidate plant genotypes using the data representations from genotypes grown in the same location and time under the same or different subfield environmental conditions.

In some embodiments, the methods and systems include establishing a supervised learning model that can be used in connection with a data representation of a pair of candidate plant genotypes to predict the relative performance for at least one phenotype of interest for the pair of plant genotypes for a given location. In some aspects, the data representations of the pair of candidate plant genotypes and/or location-specific environment is used as input into the established supervised learning model.

In some examples, methods for predicting the performance for at least one phenotype of interest for at least one pair of candidate plant genotypes includes inputting the data representations from the pair of candidate plant genotypes into the established supervised learning model. The supervised learning model may be trained to learn the genotype by environment interactions for different plant genotypes in different environments based on the underlying relationships to the data, e.g. to learn the relationship of the genotype by environment interactions with the phenotypic performance for a plurality of plant genotypes. In some aspects, the supervised learning model is trained to learn to predict performance for at least one phenotype of interest for at least one pair of candidate plant genotypes using the data representations from plants grown under the same and/or different environments.

In some examples, the candidate plant genotype is not used to establish the supervised learning or deep learning transformer model. In some aspects, the candidate plant has a simulated genotype, for example, a simulated genotype resulting from a simulated cross. In some aspects, the candidate plant genotype has an imputed or inferred genotype, for example, an inferred genotype representation based on performance data. The candidate plant genotype may be a plant at any stage of development or growth, including an immature plant or mature plant, including a plant at harvest time. Furthermore, the data representations of the candidate plants may be obtained at the same developmental or growth stage as that of the training plants used to establish the machine learning model, e.g. a supervised deep learning neural network or deep learning transformer model, or at a different developmental or growth stage as that of the plants (training or validation plants) used to establish the model.

In some examples, the methods include selecting one or more plant genotypes, based on its predicted performance for a phenotype of interest. The methods may include selecting one or more of the candidate plant genotypes, including physically selecting one or more of the candidate plants having the candidate plant genotype, having a desired predicted value, difference, probability, relative performance, average performance, or classification for the phenotype of interest.

In some examples, the established deep learning model, e.g., a deep learning transformer model or a deep learning model implementing self-attention, provides a difference, probability, relative performance, or classification for the phenotype of interest for one or more of the candidate plant genotypes as compared to a reference plant genotype. In some examples, the reference plant genotype may be a candidate plant genotype, a training plant genotype, or a plant genotype used in validation.

In some examples, the one or more candidate plant genotypes are predicted to exhibit an improved or increased desirable phenotype of interest, such as increased yield, improved agronomic trait performance such as increased drought resistance, improved standability, improved lodging resistance, improved abiotic or biotic stress resistance, and the like. In some examples, the one or more candidate plant genotypes are predicted for a phenotype of interest including but not limited to plant height, ear height, moisture, test weight, or grain yield.

In some examples, the established supervised learning model provides a predicted value for each of the candidate genotypes in the pair and assigns a classification to the predicted value, for example, a relative performance classification to indicate that one candidate plant of the pair performed better than the other. In some examples, one of the candidate plant genotypes in the pair is predicted to exhibit improved or increased desirable phenotype of interest, such as increased yield, improved agronomic trait performance such as increased drought resistance, improved standability, improved lodging resistance, improved abiotic or biotic stress resistance, and the like. In some examples, one of the candidate plant genotypes in the pair is predicted for a phenotype of interest for phenotypes including but not limited to height, ear height, moisture, test weight, or grain yield.

As an example and not by way of limitation, phenotypes of interest may include but are not limited to adjusted gross income (AGI), grain yield, yield gain, root lodging resistance, stalk lodging resistance, brittlesnap resistance, ear height, grain moisture, plant height, disease resistance, pest resistance, drought tolerance, cold tolerance, heat tolerance, salt tolerance, stress tolerance, herbicide tolerance, flowering time, color, fungal resistance, virus resistance, male sterility, female sterility, stalk strength, starch content, oil profile, amino acids balance, lysine level, methionine level, digestibility, fiber quality, plant growth, total plant area, transgene effects, response to chemical treatment, stress tolerance, gas exchange parameters, days to silk, days to shed, germination rate, biomass, dry shoot weight, nitrogen utilization efficiency, water use efficiency, relative maturity, lodging, stress emergence rate, leaf senescence rate, canopy photosynthesis rate, silk emergence rate, anthesis to silking interval, percent recurrent parent, leaf angle, leaf extension rate, chlorophyll content, leaf temperature, canopy width, leaf width ear fill, scattergrain, root mass, stalk strength, seed moisture, greensnap, shattering, visual pigment accumulation, kernels per ear, ears per plant, kernel density, kernel composition (including, but not limited to protein, oil, and/or starch composition), number of kernels per row on the ear, number of rows of kernels on the ear, kernel abortion, kernel weight, kernel size, leaf nitrogen content and grain nitrogen content, yield, including yield gain, silage yield, yield drag, abiotic traits, fertility, seedling vigor, internode length, leaf number, leaf area, tillering, brace roots, stay green, plant health, physical grain quality, or combinations thereof.

Use of the systems and methods described herein may be used to predict performance of new (untested) or previously characterized (observed) candidate plant genotypes in untested or previously characterized (observed) environments.

In some embodiments, users are presented with results, e.g., predicted performance outcome for one or more candidate plants in one or more given locations or set of locations, e.g. aggregated locations. For example, in some embodiments, input data, such as location-specific environmental data, or results for multiple locations may be aggregated, i.e. grouped together, across a plurality of locations based on shared features, such as shared environmental conditions, e.g. drought.

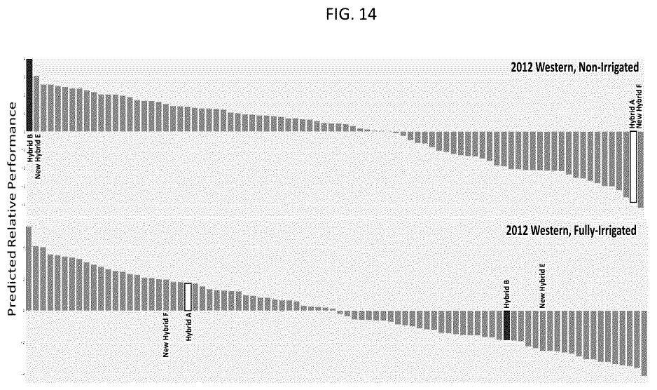

As an example and not by way of limitation, the Bradley-Terry model may be used to predict the global relative ranking of candidate genotypes based on an initial set of paired comparisons. See, for example, FIG. 14. In some embodiments, users are able to select one or more environmental variables or environmental conditions that the user is interested in, e.g. temperature, precipitation, the presence of irrigation, humidity, or evapotranspiration. The model or system can apply a ranking technique such as the Bradley-Terry model to rank the results based on the set of locations and environments, e.g., environmental variables or environmental conditions, the user has selected to rank within. In some aspects, the user may perform the ranking and select a group or subgroup of genotypes that meets the user's selection criteria.

In some aspects, the system or model may rank and provide recommendations to the user in an automated manner. Although Bradley-Terry techniques are discussed herein, any suitable ranking algorithm or approach may be used in the methods and systems described herein.

In some cases, a candidate plant genotype or set of candidate plant genotypes may be selected for product placement or advancement or inclusion in a breeding program based on its predicted performance. In some aspects, the selection may be based on whether the predicted performance, e.g. difference or probability of better performance, for a phenotype at one or more given locations, set of locations, or aggregated locations, satisfies a certain threshold value. For example, whether the candidate plant genotype meets or exceeds a desired yield amount in a drought environment or a high probability that yield will exceed that of a well-performing check variety or a reference. The threshold value may vary based on market criteria and/or the set of genetics under comparison. In some embodiments, the methods and systems may include selecting one or more candidate plants having a desired predicted difference, value, probability, or classification for one or more given locations, set of locations, or aggregated locations.

In some examples, the established supervised learning model assigns a classification to the candidate plant, for example, a winner classification, indicating that that candidate plant is predicted to perform better than another candidate plant for the phenotype of interest at a given location. In some examples, the established supervised learning model assigns a positive or negative real value to the pair of candidate plants, for example, a predicted performance difference value, indicating the difference in one candidate plant's performance versus another candidate plant's performance for the phenotype of interest at a given location.

In some embodiments, the prediction outcomes generated by the machine learning model may be used to identify which candidate plant genotypes to avoid planting in a specific environment. The modeled (learned) genotype by environment interactions may be used with candidate plants and/or locations to make predictions about performance for new (unobserved) environments and/or new (unobserved) candidate plants, or combinations thereof. In one embodiment, use of the learned genotype by environment interactions enables the comparison of existing and/or potential products' performances in environments with known environmental conditions and stress.

In some embodiments, the prediction outcomes generated by the machine learning model may be used to identify which candidate plant genotypes to recommend for planting in certain locations or environments within a single field. In one embodiment, the methods include inferring, by the machine learning model, certain environmental factors that are influential on candidate plant genotypes at certain locations, e.g. spatial units. Environment factors include but are not limited to elevation, water pooling, planting density, soil type, soil compaction, nitrogen application, previous crop rotation, and water availability.

In some embodiments, the prediction outcomes generated by the machine learning model may be used to identify candidate plant genotypes or candidate plants for use in a breeding pipeline in a breeding program. For example, some candidate plants may be used to develop products for one or more market segments and achieve breeding targets for a target population of environments for growers/farmers. In another example, candidate plant genotypes or candidate plants may be selected for inclusion in various breeding strategies based on the predictions, for example, including in the breeding pipeline or commercial production, those candidate plants predicted to have a wide adaption for performance for one or more phenotypes of interest across a set of given locations and environmental conditions. In some examples, candidate plants that perform well or are predicted to perform well in certain locations and specific environments, but not all, may be selected for use in targeted breeding strategies in a breeding pipeline or commercial production. Accordingly, the methods and systems described herein include breeding at least one of the selected candidate plants, that is, the candidate plant genotypes, for a specific environment or location based on the predicted performance of the candidate plant genotype for a phenotype of interest for one or more given locations, set of locations, or aggregated locations. One or more candidate plants or or candidate plant genotypes may be selected and/or offered as a commercial product based on the predicted performance for the phenotype of interest at one or more given locations. In some embodiments, the methods and systems described herein provide automated recommendations of candidate plants or candidate plant genotypes for use in the breeding pipeline or commercial production.

If the predictions indicate certain candidate plants or candidate plant genotypes would be a good fit for future market segments or breeding targets, these plants may be bred with at least one other plant or selfed, e.g., to create a new line or hybrid, used in recurrent selection, bulk selection, or mass selection, backcrossed, used in parent selection for making crosses, used in pedigree breeding or open pollination breeding, and/or used in genetic marker enhanced selection.

In some instances, a candidate plant with a favorable performance prediction from the machine learning model, e.g., the trained supervised model or deep learning transformer model, may be crossed with another plant or back-crossed so that a desirable genotype may be introgressed into the plant by sexual outcrossing or other conventional breeding methods. The plants may be grown and crossed according to any breeding protocol relevant to the particular breeding program. Selected candidate plants, progeny from crosses, or parts thereof may be used in a breeding pipeline in a program.

In some embodiments, unfavorable performance predictions from the machine learning model, e.g., the trained supervised model or deep learning transformer model, may be used to cull or remove candidate plants from a breeding pipeline or program if the predictions indicate the candidate plants would be a poor fit for future market segments or breeding targets.

The candidate plants may be grown in a plant growing environment, such as a greenhouse, plot, subfield, or field and further evaluated for the predicted phenotype of interest and/or additional phenotypes using any suitable techniques. In some examples, one or more grown candidate plants may be evaluated to determine if the candidate plants exhibit the predicted performance for the phenotype of interest, such as increased yield, increased drought resistance, or improved standability.

### Embodiments

The present disclosure is further illustrated in the following embodiments. It should be understood that these embodiments are given by way of illustration only.
Embodiment 1. A method for predicting the performance of a plant, the method comprising:
(a) inputting, through one or more computing devices, representations of genotypic data from two or more candidate plant genotypes and location-specific environmental data, wherein at least one of the two or more candidate plant genotypes is a reference candidate plant genotype, into a trained machine learning model, wherein the machine learning model has been trained to learn the relationship of the genotype by environment interactions with the phenotypic performance for a plurality of plant genotypes and predict performance for a phenotype of interest for one or more candidate plant genotypes compared to the reference candidate plant genotype; and
(b) generating by the trained machine model a prediction as to the performance of the one or more candidate plant genotypes for the phenotype of interest as compared to the reference candidate plant genotype at one or more locations.
Embodiment 2. The method of embodiment 1, wherein at least one of the two or more candidate plant genotypes is designated as the reference candidate plant genotype.
Embodiment 3. The method of embodiment 1, wherein the machine learning model has been trained to predict whether one or more of the candidate plant genotypes that are not the reference candidate plant genotype will perform better for the phenotype of interest compared to the reference candidate plant genotype.
Embodiment 4. The method of embodiment 1, wherein the machine learning model is trained by the method comprising:
receiving, through one or more computing devices, at least one training data set comprising representations of phenotypic performance data, genotypic data, and location-specific environmental data associated with two or more training plants at one or more locations;
inputting the representations of genotypic data and location-specific environmental data from the at least one training data set into a machine learning model; and
training the machine learning model to learn a relationship of the genotype by environment interactions with the phenotypic performance for a plurality of plant genotypes, e.g. training plant genotypes, and predict relative performance for a phenotype of interest for one or more candidate plant genotypes to create a trained machine learning model.

For example, the machine learning model may be trained to learn a relationship among the training plant genotypes and location-specific environment interactions with phenotypic performance for the plant genotypes.
Embodiment 5. The method of embodiment 4, wherein the phenotypic performance data is labeled with a binary classification of which genotype has a higher value.
Embodiment 6. The method of embodiment 1, wherein the machine learning model is trained by the method comprising:
receiving, through one or more computing devices, at least one training data set comprising representations of phenotypic performance data, genotypic data, location-specific environmental data, and spatial coordinate data associated with two or more training plant genotypes at one or more locations;
inputting the representations of genotypic data, location-specific environmental data, and spatial coordinate data from the at least one training data set into a machine learning model; and
training the machine learning model to learn a relationship of genotype by environment interactions with the phenotypic performance for the training plant genotypes and predict performance for a phenotype of interest for one or more candidate plant genotypes to create a trained machine learning model.
Embodiment 7. The method of embodiment 4 or 6, the method further comprising:
training the machine learning model to learn the relationship of genotype by environment interactions with the phenotypic performance for the plurality of training plant genotypes and predict for at least one or more spatial units comprising a plant genotype a performance for the phenotype of interest compared to a predicted performance for the phenotype of interest for at least one spatial unit of a plant genotype, e.g. a reference spatial unit.
Embodiment 8. The method of embodiment 4 or 6, further comprising:
training the machine learning model to generate a predicted performance difference in the phenotype of interest between a first candidate genotype and a second candidate genotype at a certain location.
Embodiment 9. The method of embodiment 4 or 6, further comprising:
training the machine learning model to predict a performance difference, value, probability, or classification for the phenotype of interest for one or more candidate plant genotypes for one or more given locations, set of locations, or aggregated locations.
Embodiment 10. The method of embodiment 9, wherein the one or more given locations, set of locations, or aggregated locations are represented by a single set of environmental factors for each comparison of candidate genotypes
Embodiment 11. The method of embodiment 1, the method further comprising, inputting into the trained machine learning model, through one or more computing devices, representations of spatial coordinate data for the spatial units for the two or more candidate plant genotypes.
Embodiment 12. The method of embodiment 1, where the machine learning model is trained to predict a performance for the phenotype of interest relative to one or more reference spatial units comprising at least one plant genotype, e.g, a training plant genotype, a candidate plant genotype, or a plant genotype used in validation.
Embodiment 13. The method of embodiment 1, wherein the machine learning model is a deep learning model or a supervised learning model.
Embodiment 14. The method of embodiment 1, wherein the machine learning model is a deep learning transformer model (transformer-based deep learning model) or a deep learning model implementing self-attention.
Embodiment 15. The method of embodiment 13 or 14, wherein the deep learning model uses self-attention or attention layers. In some aspects, the deep learning model uses self-attention or attention layers in its encoders and/or decoders.
Embodiment 16. The method of embodiment 1, the method further comprising, inputting into the trained machine learning model, through one or more computing devices, representations of spatial coordinate data for a given spatial unit for use in predicting how the one or more of the candidate plant genotypes will perform in the given spatial unit.
Embodiment 17. The method of embodiment 1, wherein the method further comprises generating by the trained machine learning model a prediction as to the performance of the one or more candidate plant genotypes for the phenotype of interest at a certain location as compared to the reference candidate plant genotype at the same location or different location.
Embodiment 18. The method of embodiment 1, wherein the prediction of the performance for the one or more of the candidate plant genotypes for the phenotype of interest as compared to the reference candidate plant genotype's predicted performance for the phenotype of interest is a predicted relative performance value or a predicted difference.
Embodiment 19. The method of embodiment 1, wherein the one or more of the candidate plant genotypes and the reference candidate plant genotype performances are predicted under the same environmental conditions. In some aspects, the same environmental conditions refers to same location-specific environmental conditions.
Embodiment 20. The method of embodiment 1, wherein the one or more of the candidate plant genotypes and the reference candidate plant genotype are predicted under different environmental conditions. In some aspects, the different environmental conditions refers to different location-specific environmental conditions.
Embodiment 21. The method of embodiment 1, wherein the genotype of the one or more candidate plant genotypes and the reference candidate plant genotype are the same.
Embodiment 22. The method of embodiment 1, wherein one or more candidate plant genotype and/or a reference candidate plant genotype are grown in or predicted for a spatial unit comprising a pot, row, a plot, a subfield, or a field. In some examples, the spatial unit may comprise an individual plant.
Embodiment 23. The method of embodiment 1, wherein the predicted performance of one or more of the candidate plant genotypes for the phenotype of interest for a spatial unit is compared with the predicted performance of the reference candidate plant of a different plant genotype for the same phenotype for the same type of spatial unit, e.g., a row, a plot, a subfield, or field.
Embodiment 24. The method of embodiment 1, wherein the prediction of the performance of one or more of the candidate plant genotypes for the phenotype of interest as compared to the reference candidate plant genotype's predicted performance for the phenotype of interest is a predicted probability of whether the one or more candidate plant genotypes outperforms the reference candidate plant genotype.
Embodiment 25. The method of embodiment 1, wherein the prediction of the performance for the one or more of the candidate plant genotypes for the phenotype of interest as compared to the reference candidate plant genotype's predicted performance for the phenotype of interest is a real-valued predicted relative performance value or a predicted difference at one or more given locations, set of locations, or aggregated locations.
Embodiment 26. The method of embodiment 1, wherein the trained machine learning model, e.g., a deep learning transformer-based model, makes a prediction of the relative performance for the phenotype of interest for all input candidate plant genotypes in multiple spatial locations at once.
Embodiment 27. The method of embodiment 1, wherein the prediction of the performance for each of the one or more of the selected candidate plant genotypes for the phenotype of interest is an average of the predicted performance value of that candidate plant genotype from the same or different locations and/or same or different environments.
Embodiment 28. The method of embodiment 1, wherein the prediction of the performance for the reference candidate plant genotype for the phenotype of interest is an average of the predicted performance value for the reference candidate plant genotype for same or different locations and/or same or different environments.
Embodiment 29. The method of embodiment 1, the method further comprising: generating by the trained machine learning model a prediction as to the performance of the one or more candidate plant genotypes for the phenotype of interest at a certain location as compared to the reference candidate plant genotype.
Embodiment 30. The method of embodiment 1, wherein the reference candidate plant genotype and the one or more candidate plant genotypes (non-reference candidate plant genotypes) are predicted for phenotypic performance for the phenotype of interest for the same location, e.g., in a common subfield or common field, under the same environmental conditions.
Embodiment 31. The method of embodiment 1, wherein the reference candidate plant genotype and the one or more candidate plant genotypes (non-reference candidate plant genotypes) are predicted for phenotypic performance for the phenotype of interest for plots in a common subfield or common field having the same location-specific environmental conditions.
Embodiment 32. The method of embodiment 1, wherein the reference candidate plant genotype and the one or more candidate plant genotypes (non-reference candidate plant genotypes) are predicted for performance at different locations in a common field, having different environmental conditions. In some aspects, the reference candidate plant genotype and the one or more candidate plant genotypes (non-reference candidate plant genotypes) are predicted for phenotypic performance for the phenotype of interest for plots in different subfields having the different location-specific environmental conditions but in a common field.
Embodiment 33. The method of embodiment 1, wherein the method further comprises: presenting one or more of the candidate plant genotype's predicted performance for the phenotype of interest on a user interface.
Embodiment 34. The method of embodiment 1, the method further comprising: growing one or more of the candidate plants in a spatial unit comprising a pot, a row, a plot, a subfield, or a field.
Embodiment 35. The method of embodiment 4 or 6, the method further comprising: growing one or more of the training plants in a spatial unit comprising a pot, a row, a plot, a subfield, or a field.
Embodiment 36. The method of embodiment 1, where the phenotype of interest is yield, adjusted gross income (AGI), grain yield, yield gain, root lodging resistance, stalk lodging resistance, brittlesnap resistance, plant height, ear height, grain moisture, plant height, disease resistance, pest resistance, drought tolerance, cold tolerance, heat tolerance, salt tolerance, stress tolerance, herbicide tolerance, or flowering time.
Embodiment 37. The method of embodiment 1, wherein the plant is a monocot or dicot plant.
Embodiment 38. The method of embodiment 37, wherein the plant is a soybean, maize, sorghum, cotton, canola, sunflower, rice, wheat, sugarcane, alfalfa tobacco, barley, cassava, peanuts, millet, oil palm, potatoes, rye, or a sugar beet plant.
Embodiment 39. The method of embodiment 1, wherein the location-specific environmental data comprises geographical location information, weather condition information and imagery, soil information, abiotic stress information, biotic stress information, plant growth stage information, plant developmental stage information, plant phenological stage information, planting conditions, or combinations thereof.
Embodiment 40. The method of embodiment 1, wherein the location-specific environmental data comprises phenological data for a certain location comprising average temperature, precipitation wind speed, solar radiation, photosynthetic activity, or combinations thereof.
Embodiment 41. The method of embodiment 1, wherein the environmental data comprises raw bands of electromagnetic waves as collected by the satellite.
Embodiment 42. The method of embodiment 39, wherein the abiotic stress information or data comprises one or more plant agronomic traits, row count, location irrigation, planting date, or combinations thereof and/or the biotic stress information or data comprises plant disease resistance level, e.g., for Northern Leaf Blight (NLB) and Goss's Wilt (GOSWLT), plant herbicide tolerance level, physical injuries e.g. from pathogens, herbicides, storms, plant stress.
Embodiment 43. The method of embodiment 4 or 6, the method further comprising collecting phenological data for each physiological stage of training plants in a field of plants growing at that certain location.
Embodiment 44. The method of embodiment 1, wherein the candidate plant genotype comprises a new genotype that was not used to train the machine learning model.
Embodiment 45. The method of embodiment 25, wherein the one or more given locations is a new location-specific environment that was not used to train the machine learning model
Embodiment 46. The method of embodiment 1 or 4 or 6, wherein the plant genotypes comprise genotypes from inbreds, hybrids, or varieties.
Embodiment 47. The method of embodiment 1, the method further comprising accessing a data structure comprising plant genotypes available for use in training a machine learning model or deep learning model, associated phenotypic performance data, and location-specific environmental data.
Embodiment 48. The method of embodiment 1, the method further comprising: accessing a data structure comprising candidate plant genotypes available for use in predicting performance for the phenotype of interest by a machine learning model, a supervised learning model, or a deep learning model, location-specific environmental data, and/or spatial coordinate data.
Embodiment 49. The method of embodiment 1, the method further comprising: ranking the one or more candidate plant genotypes based on their predicted performance for the phenotype of interest at one or more given locations.
Embodiment 50. The method of embodiment 1, the method further comprising selecting one or more candidate plants as a commercial product based on the candidate plant genotypes' predicted performances for the phenotype of interest at one or more given locations.
Embodiment 51. The method of embodiment 50, further comprising recommending or offering one or more candidate plants as a commercial product based on the candidate plant genotypes' predicted performance for the phenotype of interest at one or more given locations.
Embodiment 52. The method of embodiment 1, further comprising advancing in a breeding pipeline at least one of the candidate plants for a specific environment or location based on the predicted performance of the candidate plant genotype for the phenotype of interest for a given location or set of locations.
Embodiment 53. The method of embodiment 1, further comprising growing at least one of the candidate plants for a specific environment or location based on the predicted performance of the candidate plant genotype for a phenotype of interest.
Embodiment 54. The method of embodiment 4 or 6, further comprising: accessing a data structure comprising representations of data from the training data set.
Embodiment 55. A computer readable medium having stored thereon instructions to predict the performance of a plant, when executed by a processor (or computing device), cause the processor to perform the steps of any of the embodiments of embodiment 1, 4, 6, 7, 8, 9, 17, 29, 33, 47, 48, 49, 51 or 54.
Embodiment 56. A system for use in plant phenotype performance prediction comprising:
(a) one or more servers, wherein one of the servers comprises representations of plant genotypic data and location-specific environmental data associated with two or more candidate plant genotypes; and
   (b) a computing device communicatively coupled to the one or more servers, the computing device including:
   (1) a memory; and
   (2) one or more processors configured to perform operations comprising:
      (a) obtain representations of genotypic data for two or more candidate plant genotypes and location-specific environmental data at one or more locations; and
      (d) generate using a machine learning model a prediction as to the performance for one or more candidate plant genotypes for a phenotype of interest as compared to a reference candidate plant genotype at one or more locations.
Embodiment 57. The system of embodiment 56, wherein one or more servers, comprises representations of phenotypic performance data, genotypic data, and location-specific environmental data associated with two or more training plants.
Embodiment 58. The system of embodiment 56, wherein the computing device further comprises:
one or more processors configured to perform operations prior to part (b)(2)(a), the operations comprising:
   obtain at least one training data set comprising representations of genotypic data and location-specific environmental data associated with two or more training genotypes at one or more locations;
   learn genotype by environment interactions from the training data set, e.g. a relationship of genotype by environment interactions with the phenotypic performance, and generate a predicted performance for a phenotype of interest for the one or more candidate plant genotypes to create a trained machine learning model.
Embodiment 59. A system comprising:
(a) one or more servers, wherein one of the servers comprises representations of genotypic data from two or more candidate plant genotypes, location-specific environmental data, and spatial coordinate data and representations of genotypic data, location-specific environmental data, and spatial coordinate data associated with two or more training plants;
(b) a computing device communicatively coupled to the one or more servers, the computing device including:
   (1) a memory; and
   (2) one or more processors configured to perform operations comprising:
      (a) obtain at least one training data set comprising representations of genotypic data, location-specific environmental data, and spatial coordinate data associated with two or more training plant genotypes at one or more locations, and the observed phenotypic data of the genotypes in one or more location(s);
      (b) simultaneously learn from the training data set an association among training plant genotypes and location-specific environment factors to generate a relative performance prediction for a phenotype of interest for one or more plant genotypes at one or more locations using a deep learning model;
      (c) evaluate the loss function of the predicted performance associations among the plant genotypes and location-specific environment interactions with respect to their true grouping values;
      (d) adjust the weights of the deep learning model, and/or an embedding model, and/or a predictive output layer of tokens to reduce the evaluated loss; and
      (e) reiterate performing operations (a)-(d) until convergence of validation loss to a desired value.
Embodiment 60. A system comprising:
(a) one or more servers, wherein one of the servers comprises representations of genotypic data, location-specific environmental data, and spatial coordinate data from or for two or more candidate plant genotypes and representations of genotypic data, location-specific environmental data, and spatial coordinate data associated with two or more training plant genotypes;
(b) a computing device communicatively coupled to the one or more servers, the computing device including:
   (1) a memory; and
   (2) one or more processors configured to perform operations comprising:
      (a) obtain at least one training data set comprising representations of genotypic data, location-specific environmental data, and relative spatial coordinate data associated with two or more training genotypes at one or more locations;
      (b) simultaneously learn from the training data set an association among the training plant genotypes and location-specific environment factors, e.g., learn a relationship of the genotype by environment interaction with the phenotypic performance for a plurality of training plant genotypes, to generate a relative performance prediction for a phenotype of interest for one or more plant genotypes at one or more locations in a field using a deep learning model;
      (c) evaluate the loss function of the predicted performance associations among the plant genotypes and location-specific environment interactions with respect to their observed differences;
      (d) adjust the weights of the deep learning model, and/or an embedding model, and/or a predictive output layer to reduce the evaluated loss;
      (e) reiterate performing operations (a)-(d) until convergence of validation loss to a desired value; and
      (f) receive as input into the trained deep learning model representations of genotypic data for two or more candidate plant genotypes, location-specific environmental data, and spatial coordinate data as embedding vectors to generate a performance prediction for the phenotype of interest for one or more candidate plant genotypes as compared to a reference candidate plant genotype.
Embodiment 61. A system comprising:
(a) one or more servers, wherein one of the servers comprises representations of genotypic data, location-specific environmental data, and spatial coordinate data for two or more candidate plant genotypes;
(b) a computing device communicatively coupled to the one or more servers, the computing device including:
   (1) a memory; and
   (2) one or more processors configured to perform an operation comprising:
      receive as input into a trained deep learning model representations of genotypic data for two or more candidate plant genotypes, location-specific environmental data, and spatial coordinate data as embedding vectors and generate a performance prediction for a phenotype of interest for one or more candidate plant genotypes as compared to a reference candidate plant genotype.
Embodiment 62. The system of any of the embodiments of embodiment 56, 57, 58, 59, 60, or 61, wherein at least one of the two or more candidate plant genotypes is designated as the reference candidate plant genotype.
In an example, a token or embedding is provided to designate the reference plot.
Embodiment 63. The system of embodiment 61, wherein one or more of the processors is configured to perform an operation comprising: learn from the training data set to predict whether one or more of the candidate plant genotypes that are not the reference candidate plant genotype will perform better for the phenotype of interest compared to the reference candidate plant genotype.
Embodiment 64. The system of embodiment 58, wherein the phenotypic performance data is labeled.
Embodiment 65. The system of any of the embodiments of embodiment 58, 59, or 60, wherein two or more of the training plants have the same genotype.
Embodiment 66. The system of any of the embodiments of embodiment 58, 59, or 60, wherein one or more of the processors is configured to perform an operation comprising:
   learn from the training data set to predict for at least one or more spatial units comprising a plant genotype a performance for a phenotype of interest compared to a predicted performance for the phenotype of interest for at least one spatial unit of a plant genotype.
Embodiment 67. The system of any of the embodiments of embodiment 58, 59, or 60, wherein one or more of the processors is configured to perform an operation comprising:
   learn from the training data set to generate a predicted difference in the phenotype of interest between a first candidate plant and a second candidate plant at a certain location.
Embodiment 68. The system of any of the embodiments of embodiment 58, 59, or 60, wherein one or more of the processors is configured to perform an operation comprising:
   learn from the training data set to generate a predicted performance difference, value, probability, or classification for a phenotype of interest for one or more candidate plant genotypes for one or more given locations, set of locations, or aggregated locations.
Embodiment 69. The system of embodiment 68, wherein the one or more given locations, set of locations, or aggregated locations are the same or different locations.
Embodiment 70. The system of any of the embodiments of embodiment 58, 59, or 60, wherein one or more of the processors is configured to perform an operation comprising:
   learn from the training data set to generate a predicted performance for the phenotype of interest for one or more reference spatial units comprising at least one plant genotype, e.g, a training plant genotype, a candidate plant genotype, or a plant genotype used in validation.
Embodiment 71. The system of embodiment 68, wherein the machine learning model is a deep learning model or a supervised learning model.
Embodiment 72. The system of embodiment 59, 60, 61, or 71, wherein the deep learning model is a deep learning transformer model.
Embodiment 73. The system of embodiment 59, 60, 61, or 72, wherein the deep learning model uses self-attention or attention layers.
Embodiment 74. The system of embodiment 56, 60, or 61, the system further comprising one or more processors configured to perform an operation comprising:
   generate a prediction as to the performance of the one or more candidate plant genotypes for the phenotype of interest at a certain location as compared to a reference candidate plant genotype at the same location or different location.
Embodiment 75. The system of embodiment 56, 60, or 61, the system further comprising one or more processors configured to perform an operation comprising:
   generate a predicted relative performance value or a predicted difference for the one or more of the candidate plant genotypes for the phenotype of interest as compared to a reference candidate plant genotype's predicted performance for the phenotype.
Embodiment 76. The system of embodiment 56, 63, or 74, wherein the one or more of the candidate plant genotypes and the reference candidate plant genotype performances are predicted under the same environmental conditions. In one example, the reference candidate plant genotype and the one or more candidate plant genotypes (non-reference candidate plant genotypes) are predicted for phenotypic performance for the phenotype of interest for plots in a common subfield or common field having the same location-specific environmental conditions.
Embodiment 77. The system of embodiment 56, 63, or 74, wherein the one or more of the candidate plant genotypes and the reference candidate plant genotype performances are predicted under different environmental conditions. In one example, the reference candidate plant genotype and the one or more candidate plant genotypes (non-reference candidate plant genotypes) are predicted for phenotypic performance for the phenotype of interest for plots in different subfields having different location-specific environmental conditions but in a common field.
Embodiment 78. The system of embodiment 56, 63, or 74, wherein the genotype of the one or more candidate plant genotypes and the reference candidate plant genotype are the same.
Embodiment 79. The system of embodiment 56, 63, or 74, wherein one or more candidate plant genotypes and/or a reference candidate plant genotype are directed to be grown in a spatial unit comprising a pot, row, a plot, a subfield, or a field. In some examples, the spatial unit may comprise an individual plant.
Embodiment 80. The system of embodiment 56, 63, or 74, the system further comprising one or more processors configured to perform an operation comprising:
   generate a predicted performance of one or more of the candidate plant genotypes for the phenotype of interest for a spatial unit is compared with the predicted performance of the reference candidate plant of a different plant genotype for the same phenotype for the same type of spatial unit, e.g., a row, a plot, a subfield, or field.
Embodiment 81. The system of embodiment 56, 63, or 74, the system further comprising one or more processors configured to perform an operation comprising:
   generate a predicted performance for one or more of the candidate plant genotypes for the phenotype of interest as compared to the reference candidate plant genotype's predicted performance for the phenotype, wherein the predicted performance is a predicted probability of whether the one or more candidate plant genotypes outperforms the reference candidate plant genotype.
Embodiment 82. The system of embodiment 56, 63, or 74, the system further comprising one or more processors configured to perform an operation comprising:
   generate a predicted performance for the one or more of the candidate plant genotypes for the phenotype of interest as compared to the reference candidate plant genotype's predicted performance for the phenotype at one or more given locations, set of locations, or aggregated locations, wherein the predicted performance is a predicted relative performance value or a predicted difference.
Embodiment 83. The system of embodiment 60 or 61, the system further comprising one or more processors configured to perform an operation comprising:
   generate simultaneously a prediction of the relative performance for all input candidate plant genotypes for the phenotype of interest as compared to the reference candidate plant genotype's predicted performance for all input candidate plant genotypes.
Embodiment 84. The system of embodiment 56, 60, or 61, the system further comprising one or more processors configured to perform an operation comprising:
   generate a prediction of the performance for each of the one or more of the selected candidate plant genotypes for the phenotype of interest, wherein the prediction is an average of the predicted performance value of that candidate plant genotype from the same or different locations and/or same or different environments.
Embodiment 85. The system of embodiment 56, 63, 74, or 76, the system further comprising one or more processors configured to perform an operation comprising:
   generate a prediction of the performance for the reference candidate plant genotype for the phenotype of interest, wherein the predicted performance is an average of the predicted performance value for the reference candidate plant genotype for same or different locations and/or same or different environments.
Embodiment 86. The system of embodiment 56, 63, 74, or 76, the system further comprising one or more processors configured to perform an operation comprising:
   generate a prediction as to the performance of the one or more candidate plant genotypes for the phenotype of interest at a certain location as compared to a reference candidate plant genotype.
Embodiment 87. The system of embodiment 56, 63, 74, or 76, the system further comprising one or more processors configured to perform an operation comprising:
   generate a prediction for the reference candidate plant genotype and the one or more candidate plant genotypes (non-reference candidate plant genotypes) for phenotypic performance for the phenotype of interest for the same location under the same environmental conditions, for example, for plots in a common subfield or common field having the same location-specific environmental conditions
Embodiment 88. The system of embodiment 56, 63, 74, or 76, the system further comprising one or more processors configured to perform an operation comprising:
   generate a prediction for the reference candidate plant genotype and the one or more candidate plant genotypes (non-reference candidate plant genotypes) for phenotypic performance for the phenotype of interest at different locations under the same environmental conditions.
Embodiment 89. The system of embodiment 56, 63, 74, or 76, the system further comprising one or more processors configured to perform an operation comprising:
   generate a prediction for the reference candidate plant genotype and the one or more candidate plant genotypes (non-reference candidate plant genotypes) for phenotypic performance for the phenotype of interest at different locations having different environmental conditions, for example, for plots in different subfields having the different location-specific environmental conditions but in a common field.
Embodiment 90. The system of any of the embodiments of 56, or 60-89, the system further comprising one or more processors configured to perform an operation comprising:
   present one or more of the candidate plant genotype's predicted performance for the phenotype of interest on a user interface.
Embodiment 91. The system of any of the embodiments of 56-89, wherein the phenotype of interest is yield, adjusted gross income (AGI), grain yield, yield gain, root lodging resistance, stalk lodging resistance, brittlesnap resistance, ear height, grain moisture, plant height, disease resistance, pest resistance, drought tolerance, cold tolerance, heat tolerance, salt tolerance, stress tolerance, herbicide tolerance, or flowering time.
Embodiment 92. The system of any of the embodiments of 56-91, wherein the plant is a monocot or dicot plant.
Embodiment 93. The system of any of the embodiments of 56-92, wherein the plant is a soybean, maize, sorghum, cotton, canola, sunflower, rice, wheat, sugarcane, alfalfa tobacco, barley, cassava, peanuts, millet, oil palm, potatoes, rye, or a sugar beet plant.
Embodiment 94. The system of any of the embodiments of 56, 59, 60, or 61, wherein the location-specific environmental data comprises geographical location information, weather condition information and imagery, soil information, abiotic stress information, biotic stress information, plant growth stage information, plant developmental stage information, plant phenological stage information, planting conditions, or combinations thereof.
Embodiment 95. The system of any of the embodiments of 56, 59, 60, 61, or 94, wherein the location-specific environmental data comprises phenological data for a certain location comprising average temperature, precipitation wind speed, solar radiation, photosynthetic activity, or combinations thereof.
Embodiment 96. The system of any of the embodiments of 56, 59, 60, 61, 94, or 95, wherein the environmental data comprises raw bands of electromagnetic waves as collected by the satellite.
Embodiment 97. The system of embodiment of 94, wherein the abiotic stress data/information comprises one or more plant agronomic traits, row count, location irrigation, planting date, or combinations thereof and/or the biotic stress information or data comprises plant disease resistance level, e.g., for Northern Leaf Blight (NLB) and Goss's Wilt (GOSWLT), plant herbicide tolerance level, physical injuries e.g. from pathogens, herbicides, storms, plant stress.
Embodiment 98. The system of any of the embodiments of 56, 60, or 61, wherein the one or more candidate plant genotypes comprises a new genotype that was not used to train the machine learning mode or deep learning model.
Embodiment 99. The system of any of the embodiments of 68, 69, or 82, wherein the one or more given locations is a new location-specific environment that was not used to train the machine learning model or deep learning model.
Embodiment 100. The system of any of the embodiments of 56-99, wherein the training plant genotypes or training plant genotypes comprise genotypes from inbreds, hybrids, or varieties.
Embodiment 101. The system of any of the embodiments of 56, 58-61, 63, 66-68, 70, 74, 75, 80-91, wherein the phenotype of interest is plant height, moisture, grain yield, or adjusted gross income.
Embodiment 102. The system of embodiment of 56, 60, or 61, the system further comprising one or more processors configured to perform an operation comprising:
   rank the one or more candidate plant genotypes based on their predicted performance for the phenotype of interest at one or more given locations.
Embodiment 103. The system of embodiment of 56, 60, or 61, the system further comprising one or more processors configured to perform an operation comprising:
   select one or more candidate plants as a commercial product based on the candidate plant genotypes' predicted performances for the phenotype of interest at one or more given locations.
Embodiment 104. The system of embodiment of 56, 60, or 61, the system further comprising one or more processors configured to perform an operation comprising:
recommend one or more candidate plants as a commercial product based on the candidate plant genotypes' predicted performance for a phenotype of interest at one or more given locations.
Embodiment 105. The system of embodiment of 56, 60, or 61, the system further comprising one or more processors configured to perform an operation comprising:
direct at least one of the candidate plants into a breeding pipeline.
Embodiment 106. The system of embodiment of 56, 60, 61, or 105, the system further comprising one or more processors configured to perform an operation comprising:
direct at least one of the candidate plants into a breeding pipeline for a specific environment or location based on the predicted performance of the candidate plant genotype for a phenotype of interest for a given location or set of locations.
Embodiment 107. The system of embodiment of 56-61, the system further comprising
a data structure comprising plant genotypes available for use in training a machine learning model or deep learning model, associated phenotypic performance data, location-specific environmental data, and/or spatial coordinate data.
Embodiment 108. The system of embodiment of 56-61, the system further comprising
a data structure comprising candidate plant genotypes available for use in predicting performance for the phenotype of interest by a machine learning model or deep learning model, location-specific environmental data, and/or spatial coordinate data.

### EXAMPLES

The present disclosure is further illustrated in the following Examples. It should be understood that these Examples, while indicating embodiments of the invention, are given by way of illustration only. Thus, various modifications to the types of machine learning models and learned genotype by environment interactions and applying them for use in a breeding pipeline are disclosed.

### Example 1: Description of the Model and Data

Existing North American phenotypic, genotypic, and satellite-based environmental variables were combined in a deep neural network (DNN) to predict the probability that one maize hybrid will out yield another hybrid in a single location. Best linear unbiased estimators (BLUEs) for all 2012-2019 North American research experiments were used for model training and validation. BLUEs are plot-based estimates of hybrid phenotype that have been corrected for within-field error. Hybrid precommercial research experiments were considered and included early stage topcross, R-stages, and IMPACT experiments.

Genetic effects for each hybrid in the dataset were represented by observed and imputed values of each hybrid parent at approximately 24,000 current production SNPs. A variational autoencoder trained for marker platform-invariant representation of genetics was used to represent SNPs in a common latent space as described in U.S. Pat. No. 11,174,522, granted Nov. 16, 2021. Environmental data at growing locations were characterized by weather variables (temperature, precipitation, humidity, wind speed), measures of crop health (evapotranspiration, NDVI, EVI), and indicators of growing time based on a feature binning into phenological stages based on a segmentation of the seasonal EVI curve. Recorded biotic and abiotic stress data were also used to characterize research locations. The diseases considered in this study were Northern Leaf Blight (NLB) and Goss's Wilt (GOSWLT). Abiotic traits considered were early and late root lodging (ERTLPN and LRTLPN), brittlesnap (BRTSTK), row count, location irrigation, and planting date. This approach used MODIS satellite imagery to determine the phenological stage of a 15-acre region at each timepoint using the enhanced vegetative index (EVI). The classified timepoints were divided into six physiological stages: pre-emergence, green-up, post-green-up, plateau, pre-dry down, and dry down.

Average temperature, precipitation wind speed, solar radiation, and photosynthetic activity were determined for each physiological stage by combining stage with gridded weather data from GRIDMet.

### Example 2: Model Validation

The ability of the DNN to predict the probability that one hybrid will out yield another within a specific location was validated using three different approaches. In the first approach, the DNN was trained using 2012-2019 data, except for a held out set of random hybrids. Predictive accuracy of the testing set of hybrids within this model provided an assessment of the model's ability to generalize to unobserved genotypes in historical training locations. The second validation approach assessed the ability of the DNN to predict the performance of hybrids and environments not included in model training. To accomplish this, the DNN was trained using 2012-2018 data and used to predict the performance of 2019 hybrids in 2019 locations. Hybrids tested in 2019 were removed from the 2012-2018 training dataset to ensure that predictive performance reflected the expected generalization to both new genotypes and novel environments. Finally, the ability of the model to predict the performance of the 2019 hybrids-representing novel genetics—in previously observed environments was determined by predicting the held-out 2019 hybrids in 2012-2018 locations. Hybrids found in 2019 and previous years were used to compare predictions directly with observed values.

In each validation case, a series of hybrid pairs and locations were selected, and the model used to determine the probability that the first hybrid would out yield the second hybrid in a specific location. Hybrid pairs were considered for comparison if they had 30 locations in common and each individual hybrid was found in at least 100 locations. The predicted probability was then compared to the observed yield difference between the two hybrids to assess model accuracy.

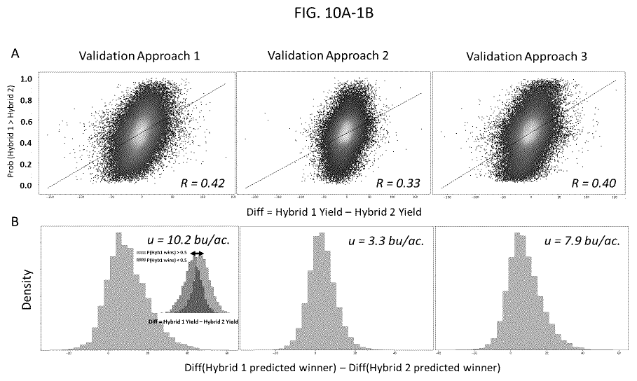

FIG. 10A demonstrates the correlation between the predicted probability of hybrid 1 out yielding hybrid 2 in a given location versus the observed yield difference for each validation approach. A significant, moderate positive correlation was observed for all validation approaches, ranging from 0.32-0.42. This magnitude of correlation is in line with expectations given the known heritability of maize grain yield among North American Pioneer hybrids. The lowest correlation was observed for the validation approach that removed all 2019 locations and hybrids from training. However, the correlation of 0.32 is significant and is comparable to the mean correlation for yield in whole genome prediction applications using multiple locations. We observed a correlation of 0.39 for the validation approach where new 2019 hybrids were predicted into known environments from 2012-2018. This value is similar in magnitude to the mean correlation observed when all data is used in the training, showing that new genetics can be predicted directly within locations under known environmental and biotic/abiotic stress.

To distinguish the impact of predicted GxE from main genetic effects, the difference in observed BLUEs between a hybrid pair was determined for locations where the probability of hybrid 1 out yielding hybrid 2 was greater than 0.5. This value should be positive if the model accurately predicts GxE interactions between hybrid pairs. FIG. 10B shows the distribution of differences across all testing hybrid pairs that had at least 5 locations where each hybrid was predicted to beat the other. In each case, a significant positive difference is observed, indicating that the DNN is accurately predicting GxE interactions.

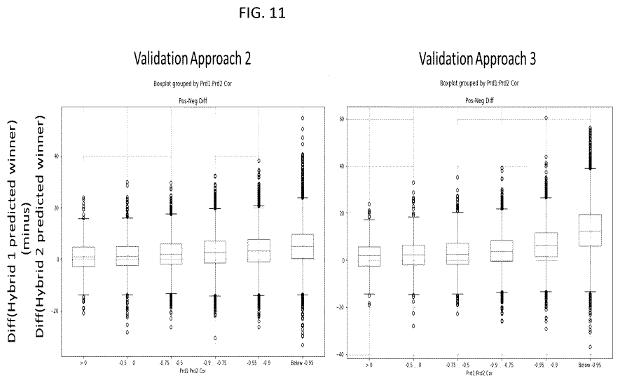

One factor limiting predictive power within the model is the actual presence of repeatable GxE influencing the prediction. Considering 2 hybrids, X and Y, the DNN model provides an implicit indicator of predictive repeatability in the form of the correlation between the predictions of X as hybrid 1 and Y as hybrid 2 vs. Y as hybrid 1 and X as hybrid 2. Higher consistency of the GxE predictions is indicated by this prd1:prd2 correlation approaching −1, while correlation values significantly greater than −1 indicate that differences in predictions among locations are driven by predictive noise rather than repeatable GxE signal. FIG. 11 shows the association between the GxE predictive repeatability and the observed yield BLUE difference score from FIG. 10B for validation approaches 2 and 3. These show that the predictive repeatability serves as a reliable indicator of true GxE signal for unobserved genetics in both historical (validation approach 3) and novel (validation approach 2) environments.

### Example 3: Product Placement and Breeding for Specific Environments

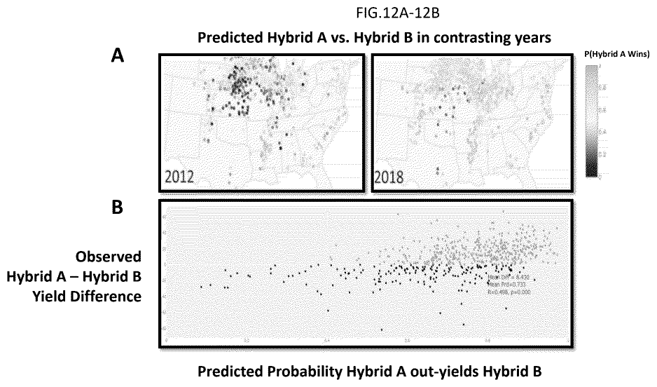

The DNN can be used to predict and compare existing and/or potential products in previous sets of environments with known environmental conditions and stress. FIG. 12A compares the predicted performance of Hybrid A and Hybrid B in all 2012 and 2018 research locations. Hybrid A is a high yielding line with low drought resistance, while Hybrid B is a product bred for drought resistance. The positive correlation (FIG. 12B) between the probability of Hybrid A out yielding Hybrid B and observed yield in locations containing both hybrids shows the model is accurately predicting GxE. FIG. 12A shows that Hybrid A has a high probability to out yield Hybrid B in most environments. However, in locations with low yields or recorded drought stress, Hybrid B has a much higher probability of out yielding Hybrid A. This is most evident in 2012, a year with severe drought pressure wherein Hybrid B is predicted to out-yield Hybrid A at most non-irrigated locations throughout midwestern and western regions. In non-drought years, such as 2018, Hybrid B is predicted to out-yield Hybrid A primarily in non-irrigated Western locations with drought stress.

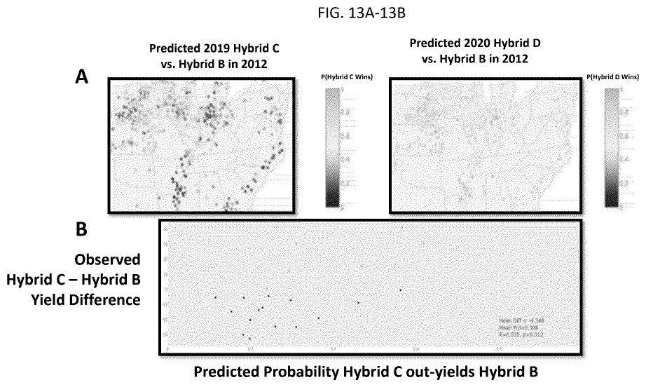

The DNN uses genetic and location-specific environmental data to make performance predictions and may be used to predict G×E patterns on untested, genotyped material. For example, Hybrid C, a 2019 R2 hybrid, shows a similar performance pattern as Hybrid B when compared to Hybrid A in 2012 western, non-irrigated environments (FIG. 13A), while maintaining high performance compared to Hybrid A in 2019 western drought stress environments (FIG. 13B). Hybrid D, a 2020 far Western early-stage hybrid with a new code parent is predicted to outperform Hybrid A throughout a broad set of 2012 environments (FIG. 13A), indicating it has drought stress potential.

In addition to providing predictions of head-to-head comparisons, the DNN model may be used to rank collections of hybrids in environments of interest, which was accomplished using the Bradley-Terry model. FIG. 14 shows an example, wherein pairwise predictions were reconciled into preference values for far Western pre-commercial hybrids in a set of irrigated and non-irrigated 2012 Western environments. Within the non-irrigated environments, Hybrid B is predicted to outperform all hybrids, with Hybrid E predicted best among the 2020 class. The otherwise high-yielding genetics of Hybrid A and Hybrid F are ranked at the bottom of this hybrid list. However, within the same region and year, the ranks partially reverse order under Irrigation, with Hybrid A and Hybrid F in the top half and Hybrid B and Hybrid E in the bottom half.

### Example 4: Data Collection and Preprocessing

Production BLUEs for all North American research locations, encompassing early and late-stage testing, from 2012-2019 were retrieved from an internal database, along with the production SNP sets for all F1 parents. Missing SNPs present within the parents were imputed using parent-progeny imputation via the posterior decoding algorithm of a Hidden Markov Model. See, for example, U.S. Pat. No. 11,174,522, granted Nov. 16, 2021. All SNPs were represented using a 2-channel encoding scheme, wherein the first channel indicated the probability of alternative homozygous genotypes, with values of −1 and 1 indicating certainty in alternative homozygous states and values in between representing uncertainty as given by posterior decoding. The second channel denoted whether the genotype was missing, observed/imputed, or observed heterozygous with values −1, 0, and 1, respectively. Following SNP retrieval, parental SNPs were transformed into a set of continuous latent vectors corresponding to the space used for bridge imputation. Briefly, we used a set of encoders consisting of an initial locally-connected 1D convolutional layer followed by 2 layers of shared-filter 1D convolutions and max pooling, prior to flattening and running through a densely-connected layer. All layers used the MiSH activation function, with dropout rates of 0.1 between each layer. The final output was based on a variational autoencoder (VAE) architecture, wherein each encoder produced a representation of a multi-dimensional mean and standard deviation within the latent space. The set of latent vectors included a 64-dimensional “global” encoding, trained with the VAE architecture to reconstruct the full set of production SNPs. It also included 28 64-dimensional “local” encodings, each trained using the VAE architecture to encode 100 cM regions in order to reconstruct the underlying set of high-density SNPs in the region, based on whole genome sequencing.

Environmental data from each location was retrieved from an internal database (db). Each variable was quantile-normalized prior to training to ensure even scaling and approximately normal distributions of each environmental input variable. Additionally, values for row count and planting date were retrieved and scaled to between 0 and 1 based on their theoretical maxima. Location-values for damage traits—natural brittlesnap, early root lodging, late root lodging, Goss' Wilt, and Northern Leaf Blight—were calculated based on the mean experimental unit value for measured plots at the location of interest. If the damage trait was not measured for a given location, then the maximum value for that trait (indicative of no damage) was used. These values were scaled between 0 and 1 based on their theoretical maxima.

### Example 5: Predictor Architecture and Training

Prior to training, the training, validation, and testing sets of hybrids were defined. The testing sets were set aside for evaluation, while the training set was used for direct DNN optimization and the validation set was used to assess the relative quality of the model at each epoch. For the training with all years included, mutually-exclusive testing and validation sets were defined by choosing every 7th location and randomly selecting hybrids such that at least 5 hybrids from each location would be present within the validation set and 5 would be present within the testing set, enriching for the presence of hybrids in multiple locations to assess prediction of GxE across multiple environments. For the hold-1-year-out training scheme, all hybrids within a given year were held-out for testing, including from previous years. Furthermore, any hybrids from previous years containing precodes in the testing year were held out.

In this embodiment, the DNN predictor architecture consists of 3 components: a genotype embedder, an environmental embedder, and a final predictor. This 3-part architecture reduces the computational complexity of predictions during runtime, as the genotype and environmental embeddings only need to be computed once for each hybrid and for each environment rather than for every new combination. The genotype embedder receives the global and local latent vectors for a given hybrid, with the hybrid vectors constructed via the concatenation of the F1 parents' vectors. Two initial fully connected layers within the genotype embedder process the global vectors alone, while a second set separately processes the concatenation of the global and local vectors. The resulting intermediate vectors based on only global and on local and global are concatenated prior to 2 additional layers of fully-connected layers, leading to a 512-dimensional embedding. As with the latent encoders, the MiSH activation function is used throughout, with a 0.1 dropout rate between all layers. A separate environmental embedder receives the input from the phenological model, and this is run through 2 fully connected layers, with batch normalization of the input and the first intermediate layer. A dropout rate of 0.5 is applied following the second batch normalization layer, and the MiSH activation function is used throughout. The final environmental embedding size is 512, equal to the hybrid embedding vector size. The final predictor receives an embedding of the 2 hybrids to compare, along with the environmental embedding. The concatenation of these vectors is processed by 2 fully-connected layers with 0.1 dropout rates and MiSH activation functions, and a final single neuron layer contains a sigmoid activation function to produce a value between 0 and 1.

During training, locations were divided into groups based on their plot row counts (2, 4, 8) and commercial check yield (below 150, 150-200, 200+). This was done to ensure sufficient representation of stressful, moderately-stressful, and non-stress environments with a variety of plant-plant competition levels. Each combination of row count and commercial check yield was evenly sampled within each minibatch of size 96. Within each group, locations were sampled in proportion to their hybrid counts. Random pairs of hybrids within the location of interest were selected for presentation during training, with the target response set as 1 if the first hybrid out-yielded the second and 0 otherwise. The specific hybrid designated as hybrid 1 was also randomly selected during sampling. Optimization proceeded using the Ranger version of stochastic gradient descent, with the binary cross entropy loss function. See, for example, lessw2020/Ranger-Deep-Learning-Optimizer on github on the world wide web.

### Example 6: Model Evaluation and Interrogation

During the prediction stage, each hybrid pair was evaluated bi-directionally (i.e. hybrid A in slot 1/hybrid B in slot 2 [prd1], and hybrid B in slot 1/hybrid A in slot 2 [prd2]). The final prediction for a given hybrid in a given location was based on the averaged predicted probability that hybrid A out-yields hybrid B, calculated a (prd1+(1−prd2))/2. The bi-directional predictions also allowed calculation of the prd1:prd2 correlation over locations, a measure of predictive repeatability.

Shapley Additive explanations (SHAP) plots for hybrid comparisons were made using a fast approximation. A gradient-boosted trees model was fit to the full set of predictions for a hybrid pair with XgBoost, using a 85:15 train:validation split for early stopping. SHAP evaluation was then conducted on the boosted trees model using the corresponding Python library. See, for example, slundberg/shap on github on the world wide web.

Rankings of multiple hybrids across locations were conducted using model-based reconciliation of the initial pairwise rankings. For each location, each hybrid was paired with 30 random hybrids for pairwise prediction. Predictions across all hybrids and all locations were then reconciled into predicted preferences using the Bradley-Terry model as implemented in the choix Python library. See, for example, choix on the world wide web.

### Example 7: Prediction of Continuous Phenotype Differences

Most phenotypes of interest (e.g. plant height, moisture, grain yield) occur on a continuous, metric scale. Therefore, in addition to predicting the probability of one genotype outperforming another, one may alternately or additionally predict the difference in performance between the two genotypes. In some examples, a signed difference in performance is predicted. For instance, if Hybrid B performs 5 bushels/acre better than Hybrid A, the absolute difference would be 5, but the signed difference of A−B would be −5. The signed difference thereby provides relative performance and direction. This analysis leveraged the continuity of the original grain yield phenotype to train a predictor of the difference in grain yield for two hybrid corn genotypes at a given location. Production BLUEs and markers were obtained as in Example 1, with the variational global and local encoders used to project hybrid parental genotypes into the genetic latent space.

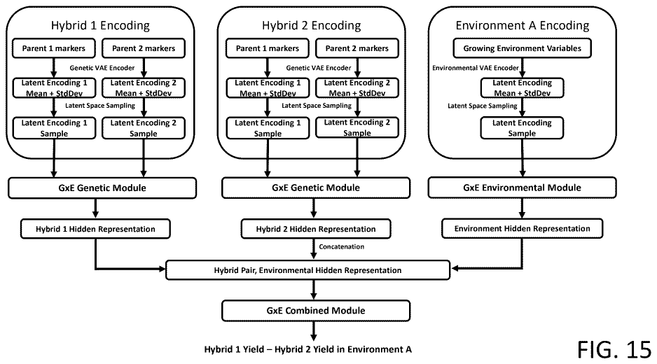

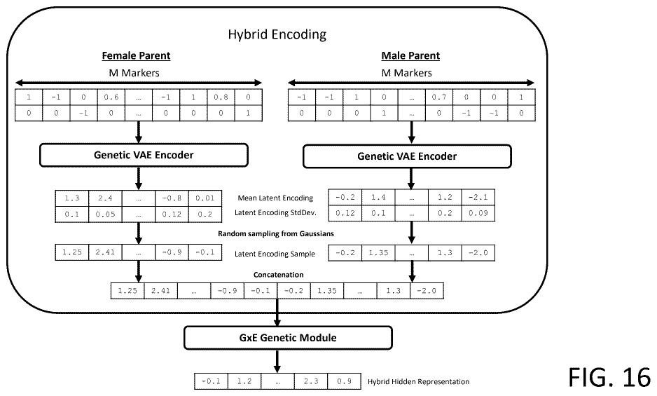

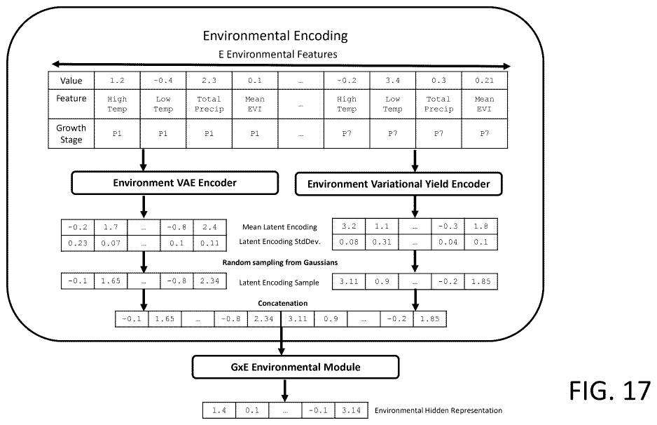

In addition to the genetic latent space, an environmental latent space was constructed using a separate variational autoencoder framework in a separate pre-training step. The purpose of this encoder was to capture the relevant environmental variance at many locations, with a reduced set of features. Environmental data was retrieved at 250 m resolution for approximately 900,000 commercial grower corn locations with combine yield data available between 2008 and 2020. The enhanced vegetative index (EVI) derived from the MODIS 250 m satellite imagery to assign growth stages to each location/growing season combination, and summary statistics of precipitation, temperature, humidity, evapotranspiration, normalized difference water index (NDWI), normalized difference vegetative index (NDVI), and EVI were provided for each of 7 assigned growth stages. Individual summary statistic variables were then clipped and transformed to conform to an approximately unimodal, symmetric distribution around zero. Examples of the preprocessing and encoding of the genetic and environmental variables may be found in FIGS. 15-17.

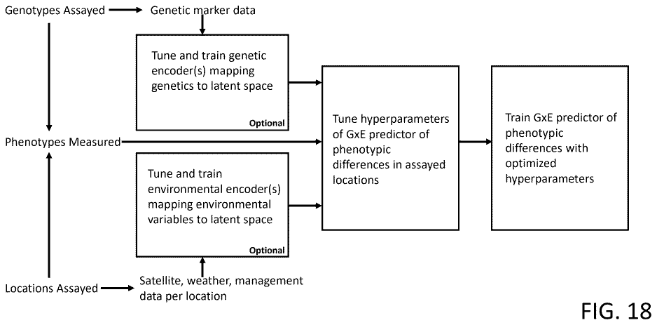

Following environmental variable transformation and normalization, hyperparameter tuning was used to find the optimal neural network structure and training parameters to achieve minimal loss using the standard VAE loss objective. Standard normal distributions were assumed for both the observed and latent variables, with the original input variables providing the target for the reconstruction error term. The base VAE structure consisted of dense feed-forward layers for both the encoder and decoder, while tuning variables included the batch size, learning rate, number of hidden layers, hidden layer dimensionality, latent space dimensionality, the presence of batch normalization, and the dropout rate. The Asynchronous Successive Halving Algorithm (ASHA) was used for efficient exploration of the parameter space, with 500 total experiments. To encourage the environmental encoding to highlight agronomically-relevant variation, a second variational encoder was fit to the environmental data, with the target output consisting of the mean yield in the given grower's field. Tuning parameters and neural network structures were consistent with the first environmental VAE, but the target output only consisted of the scalar target yield instead of the original environmental features. Following tuning of both environmental encoders and decoders with respect to the variational VAE loss function, each network was trained using its optimal hyperparameters. The overall process of training and tuning is outlined in FIG. 18.

Once the environmental encoders were trained, the hyperparameter tuning was performed on the GxE deep learning predictor structure, e.g. the deep NN. The weights of the genetic and environmental encoder neural networks were held fixed and used for pre-processing the marker data and the environmental data, respectively, into latent vectors. Stochasticity was added to this process by sampling from the genetic and environmental latent spaces for each input, rather than using only the mean latent vectors. The general structure of the GxE predictor network reflected that of Example 1, with an input for each of the two hybrid genotypes, an input for the environment for which to predict their relative performance, and an output for the difference between the two genotypes in the given environment. Following the separate inputs, dense layers were used to process each input separately, followed by concatenation of the resulting intermediate values before combined process by a set of hidden dense layers on the combined vectors. Hyperparameter tuning based on minimization of the average loss for two different held-out years —2012 and 2018—was used to choose the optimal hyperparameters for this structure. These hyperparameters included the learning rate, batch size, the use of batch normalization, the number and sizes of genetic and environmental hidden layers, and number and sizes of post-concatenation layers, and the amount of dropout and weight decay. The configuration with the optimal hyperparameters was then trained for 250 epochs of 200,000 samples with 2018 predictive performance used for selection of the best early stopping point.

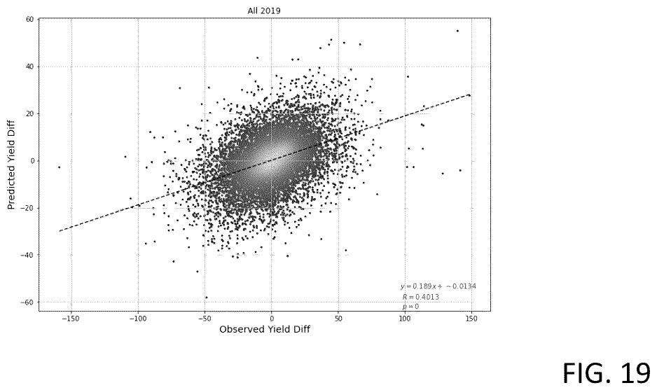

The year 2019 was held out of all prior training and validation steps to serve as a test set. Evaluation of the test set showed a Pearson correlation of 0.4 between observed and predicted yield differences across all evaluated pairs (FIG. 19). Comparisons of individual hybrid pairs for which there were at least 5 testing locations where each hybrid in the pair was shown to be superior were also evaluated. This type of evaluation allowed specific quantification of the ability to detect G×E patterns, as the genetic comparison remains constant across locations. Across testing locations, the genotype with the higher predicted performance had—on average—a 4.6 bu/ac yield advantage and was found to beat the contrasting in 75% of cases. Moreover, for a given hybrid pair, the Spearman rank correlation between the predicted and observed yield differences averaged 0.124 and was positive 80% of the time, showing the potential for ranking locations by G×E patterns.

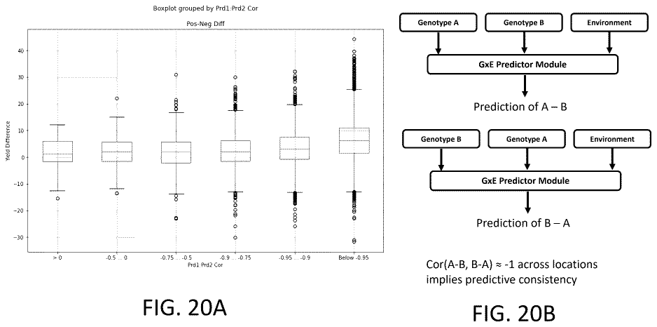

The invariance of predictions to order of presentation to the GxE predictor provided an additional score that was used to determine reliability. For any given pair of genotypes—A and B—to compare, their inputs may be provided to the predictor, e.g. the DNN, to predict the difference A−B or the difference B−A. When predicted both ways across a set of locations, a correlation of −1 between the two prediction sets would denote perfect consistency, while a value at 0 would indicate no consistency. Across the testing data, 49.75% of genotype pairs had a correlation between −1 and −0.95, while only 6.8% of pairs had correlations above −0.5. In accordance with the relationship of predictive invariance to predictive reliability, genotype pairs with consistency scores between −1 and −0.95 averaged a 6.5 bu/ac difference between the genotype with the higher vs. lower predicted yield and had a mean Spearman rank correlation of 0.174 between observed and predicted differences across location (FIG. 20). By contrast, genotype pairs with consistency scores above −0.5 consistently averaged less than a 2 bu/ac yield advantage to the predicted winner, with Spearman rank correlations among locations all below 0.07.

### Example 8: Adjusted Gross Income

The predictor structure, e.g. the deep NN, provided for all examples may generalize to any phenotype of interest. Therefore, it was used to predict additional traits that define optimal growing zones for testing or product placement. The adjusted gross income (AGI) may be a particular phenotype of interest, as it defines the expected income per unit land. For a given grain yield (Y) and grain moisture (M), AGI was calculated as follows:

$$Y ( {Y}_{p} - D ( M - {M}_{*} ) )$$

where Y is the measured grain yield, Y_P is the price per unit of yield, D is the dry down cost, M is the measured grain moisture, and M_* is the moisture required prior to sale. If M>M_* at harvest, the difference is assumed to be 0. Taking the difference between the AGIs of two genotypes at a given location, it can be written as:

$${AGI}_{1} - {AGI}_{2} = {Y}_{p} ( {Y}_{1} - {Y}_{2} ) - D ( {Y}_{1} {M}_{1} - {Y}_{2} {M}_{2} )$$

Where (1) and (2) denote the two genotypes in the comparison, Y_X denotes the grain yield of hybrid X, and M_X denotes the grain moisture of hybrid X.

Training of the AGI predictor leveraged 2 neural networks, both the same structure as in Example 1, with the output given by a linear activation function as in Example 2. The first neural network was trained to predict the continuous difference (Y₁−Y₂), while the second neural network was trained to predict the second continuous difference of (Y₁M₁−Y₂M₂).

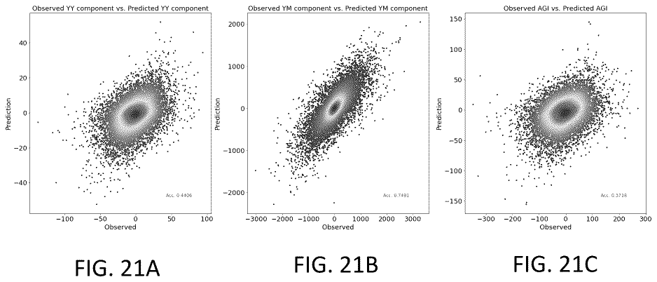

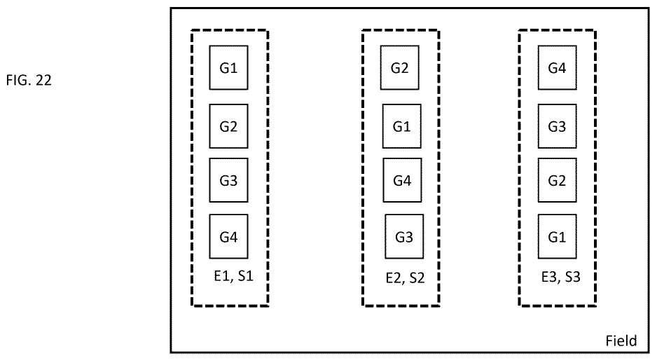

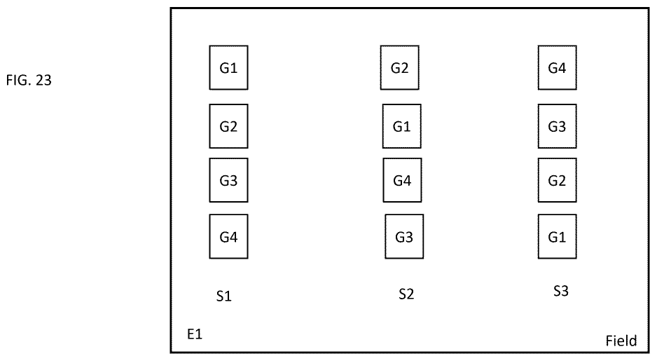

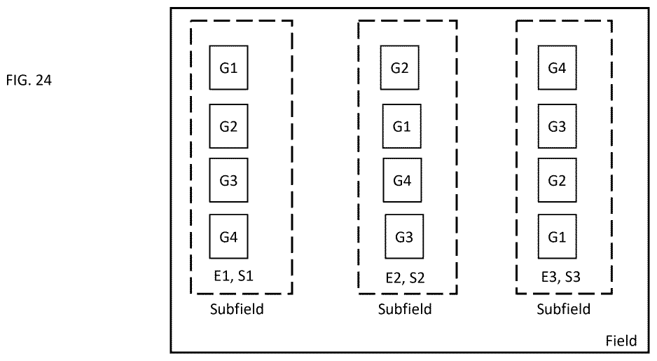

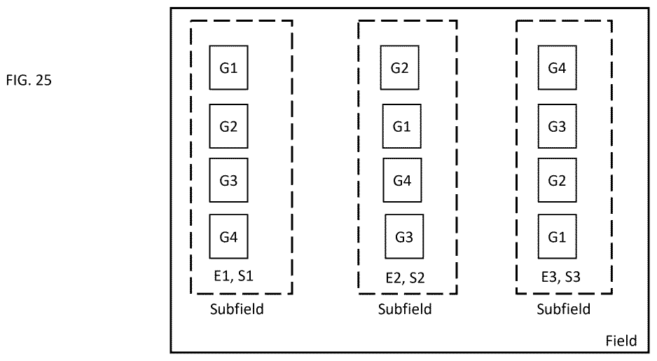

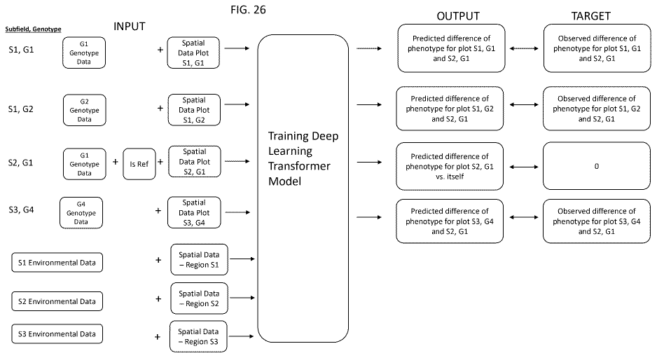

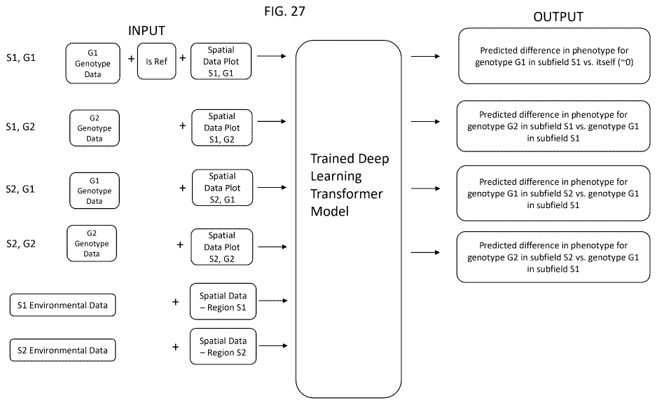

Combining both of these into the AGI difference equation then yields the AGI difference in income/unit land area (e.g. dollars per acre). Following of both neural networks, the predictability of the yield difference, the yield x moisture difference, and the full AGI difference was evaluated on a held out set of locations across years. The overall Pearson correlation was 0.44 for predicted vs. observed yield differences and 0.75 for the product of yield and moisture (FIG. 21). When substituted into the AGI difference equation, the predicted vs. observed AGI difference was found to be 0.37, similar in magnitude to yield predicted vs. observed correlations.

### Example 9: Deep Learning Transformer Model

The general methodology described herein of using a deep learning algorithm for integrating genetic and environmental representations to make direct predictions of relative performance can be generalized to environmental-contextualized comparisons of more than two genotypes. Deep neural networks implementing self-attention, such as transformers, provide a means for generalizing the integration of multiple sources of information when making context-informed predictions. By applying these models to the prediction of genotype performance under the influence of GxE interactions, one can simultaneously obtain the expected relative performance for K>=2 genotypes within an environment while accounting for field-level environmental factors and subfield-level spatial trends.

A transformer based neural network for predicting GxE interactions will have two primary components as input, corresponding to genotypes and environments. Each genotype will be encoded as a latent vector representation based on marker information. Hybrid genotypes are based on the concatenation of the two parents' embeddings, while varietal genotypes use only the embeddings conditioned on the variety's markers. The concatenation of global and local latent genotype vectors, based on pre-trained encoders, provides the initial input. One or more initial fully connected, trainable layers is used to transform these latent genotype vectors into the standard D-dimensional input size for the transformer layers. Likewise, an environmental vector based on a pre-trained encoder is transformed into a D-dimensional vector representing the growing environment of a field.

During training, two or more genotypic embeddings, along with at least one environmental embedding for a field, are input into the neural network. If the environmental conditions are assumed to be uniform within a field, the growing environment will only be represented with a single embedding. However, subfield variation may be represented by including multiple environmental embeddings, each corresponding to a specific section of the field.

To account for both environmental spatial variation and genotype-specific plant-plant competition effects that can occur within the field, learnable spatial embeddings are arithmetically added to both the genotype embeddings and any subfield environmental embeddings prior to their input into the first transformer layer. These spatial transformations transform a (row, column) coordinate for a field plot into a D-dimensional vector. For subfield regions, the field coordinate corresponding to the region's centroid may be passed as input. Geometrically, the spatial embeddings should be approximately invariant to rotations and translations of the full coordinate system. During training, as part of the data augmentation strategy, random rotation and translation transformations are made to the row-column coordinates for each sample input. This allows the network to learn representations of how plots in the field are position relative to one another and to the subfield region, regardless of how the field is oriented in absolute space.

For each input set of genotypes and environments, a single genotype is selected to serve as the reference. The reference genotype is assigned a value of 0 for the prediction, and this also indicates that all output predictions for non-reference genotypes should be made relative to the reference. For example, assuming that within an environment genotypes A, B, and C had trait values of 100, 150, and 200, the relative trait values would be A=0, B=50, C=100 if A were designated as the reference but would be A=−50, B=0, C=50 if B were designated the reference. The reference designation is provided to the deep neural network through a learnable D-dimensional embedding that is arithmetically added to the genotype and spatial embeddings. During training, the reference genotype is chosen randomly for each sample. This type of data augmentation thereby encourages learning how trait values of the genotypes relate to one another in a given environment, which is the generalized version of the pairwise approach described in previous examples.

Training proceeds by inputting genotype and location-specific environmental data from previous field experiments into the deep neural network, with embeddings of spatial information and reference designations added as previously described. Predictions of relative performance are obtained for each of the input genotypes, comparing the predicted to the true relative performance observed for the plot of a genotype within the environment. The weights of the neural network may then be updated based on the loss calculated from the comparison of true and predicted relative values. Training may proceed until approximate convergence of prediction error on a validation set of data is achieved. Following the completion of training, new combinations of genotypic, environmental, and/or spatial coordinate data can be input into the deep neural network in order to simultaneously obtain predictions of relative performance for all input genotypes at once.

1. A method for predicting the performance of a plant, the method comprising:
(a) inputting, through one or more computing devices, representations of genotypic data from two or more candidate plant genotypes and location-specific environmental data, wherein at least one of the two or more candidate plant genotypes is a reference candidate plant genotype, into a trained machine learning model, wherein the machine learning model has been trained to learn a relationship of genotype by environment interactions with the phenotypic performance for a plurality of plant genotypes and predict performance for a phenotype of interest for one or more candidate plant genotypes compared to the reference candidate plant genotype; and
(b) generating by the trained machine model a prediction as to the performance of the one or more candidate plant genotypes for the phenotype of interest as compared to the reference candidate plant genotype.

2. The method of claim 1, wherein the machine learning model has been trained to predict whether one or more of the candidate plant genotypes that are not the reference candidate plant genotype will perform better for the phenotype of interest compared to the reference candidate plant genotype.

3. The method of claim 1, wherein the machine learning model is trained by the method comprising:
receiving, through one or more computing devices, at least one training data set comprising representations of genotypic data and location-specific environmental data associated with two or more training plants at one or more locations;
inputting the representations of data from the at least one training data set into a machine learning model; and
training the machine learning model to learn a relationship of genotype by environment interactions with the phenotypic performance for a plurality of training plant genotypes and predict performance for a phenotype of interest for one or more candidate plant genotypes to create a trained machine learning model.

4. The method of claim 1, the method further comprising:
receiving, through one or more computing devices, at least one training data set comprising representations of genotypic data, location-specific environmental data, and spatial coordinate data associated with two or more training plants at one or more locations;
inputting the representations of genotypic data, location-specific environmental data, and spatial coordinate data from the at least one training data set into a machine learning model; and
training the machine learning model to learn a relationship of genotype by environment interactions with the phenotypic performance for a plurality of plant genotypes and predict performance for a phenotype of interest for one or more candidate plant genotypes to create a trained machine learning model.

5. The method of claim 3 or 4, the method further comprising:
(a) training the machine learning model to learn the relationship of genotype by environment interactions with the phenotypic performance for the plurality of plant genotypes and predict for at least one or more spatial units comprising a plant genotype a performance for a phenotype of interest compared to a predicted performance for the phenotype of interest for at least one spatial unit of a plant genotype; or
(b) training the machine learning model to generate a predicted difference in the phenotype of interest between a first candidate plant and a second candidate plant at a certain location; or
(c) training the machine learning model to predict a performance difference, value, probability, or classification for a phenotype of interest for one or more candidate plant genotypes for one or more given locations, set of locations, or aggregated locations.

6. The method of claim 1, the method further comprising, inputting into the trained machine learning model, through one or more computing devices, representations of spatial coordinate data for the spatial units for the two or more candidate plant genotypes; or inputting into the trained machine learning model, through one or more computing devices, spatial coordinate data for a given spatial unit for use in predicting how the one or more of the candidate plant genotypes will perform in the given spatial unit.

7. The method of claim 1, wherein the machine learning model is a deep learning model or a supervised learning model.

8. The method of claim 1, wherein the method further comprises generating by the trained machine learning model a prediction as to the performance of the one or more candidate plant genotypes for the phenotype of interest at a certain location as compared to the reference candidate plant genotype at the same location or different location.

9. The method of claim 1, wherein the prediction of the performance for the one or more of the candidate plant genotypes for the phenotype of interest as compared to the reference candidate plant genotype's predicted performance for the phenotype is a predicted relative performance value or a predicted difference; or
wherein the predicted performance of one or more of the candidate plant genotypes for the phenotype of interest for a spatial unit is compared with the predicted performance of the reference candidate plant of a different plant genotype for the same phenotype for the same type of spatial unit; or
wherein the prediction of the performance of one or more of the candidate plant genotypes for the phenotype of interest as compared to the reference candidate plant genotype's predicted performance for the phenotype is a predicted probability of whether the one or more candidate plant genotypes outperforms the reference candidate plant genotype; or
wherein the prediction of the performance for the one or more of the candidate plant genotypes for the phenotype of interest as compared to the reference candidate plant genotype's predicted performance for the phenotype is a predicted relative performance value or a predicted difference at one or more given locations, set of locations, or aggregated locations; or
wherein the prediction of the performance for each of the one or more of the selected candidate plant genotypes for the phenotype of interest is an average of the predicted performance value of that candidate plant genotype from the same or different locations and/or same or different environments; or
wherein the prediction of the performance for the reference candidate plant genotype for the phenotype of interest is an average of the predicted performance value for the reference candidate plant genotype for same or different locations and/or same or different environments.

10. The method of claim 1, wherein the one or more of the candidate plant genotypes and the reference candidate plant genotype performances are predicted under the same environmental conditions; or
wherein the one or more of the candidate plant genotypes and the reference candidate plant genotype are predicted under the different environmental conditions; or
wherein the reference candidate plant genotype and the one or more candidate plant genotypes (non-reference candidate plant genotypes) are predicted for phenotypic performance for the phenotype of interest for the same location under the same environmental conditions; or
wherein the reference candidate plant genotype and the one or more candidate plant genotypes (non-reference candidate plant genotypes) are predicted for their performance at different locations under the same environmental conditions; or
wherein the reference candidate plant genotype and the one or more candidate plant genotypes (non-reference candidate plant genotypes) are predicted for performance at different locations having different environmental conditions.

11. The method of claim 1, the method further comprising: selecting one or more of the candidate plant genotypes and growing one or more candidate plants in a spatial unit comprising a pot, a row, a plot, a subfield, or a field.

12. The method of claim 1, wherein the phenotype of interest is yield, adjusted gross income (AGI), grain yield, yield gain, root lodging resistance, stalk lodging resistance, brittlesnap resistance, ear height, grain moisture, plant height, disease resistance, pest resistance, drought tolerance, cold tolerance, heat tolerance, salt tolerance, stress tolerance, herbicide tolerance, or flowering time.

13. (canceled)

14. (canceled)

15. The method of claim 1, wherein the location-specific environmental data comprises geographical location information, weather condition information and imagery, soil information, abiotic stress information, biotic stress information, plant growth stage information, plant developmental stage information, plant phenological stage information, planting conditions, or combinations thereof.

16. The method of claim 1, further comprising breeding at least one of the candidate plants for a specific environment or location based on the predicted performance of the candidate plant genotype for the phenotype of interest for one or more given locations, set of locations, or aggregated locations.

17. A computer readable medium having stored thereon instructions to predict the performance of a plant, when executed by a processor (or computing device), cause the processor to perform the steps of claim 1.

18. A system comprising:
(a) one or more servers, wherein one of the servers comprises representations of genotypic data and location-specific environmental data associated with two or more candidate plant genotypes; and
(b) a computing device communicatively coupled to the one or more servers, the computing device including:
(1) a memory; and
(2) one or more processors configured to perform operations comprising:
(a) obtain representations of genotypic data for two or more candidate plant genotypes and location-specific environmental data at one or more locations; and
(d) generate using a machine learning model a prediction as to the performance for one or more candidate plant genotypes for a phenotype of interest as compared to a reference candidate plant genotype at the one or more locations.

19. The system of embodiment 18, wherein one or more servers, comprises representations of phenotypic performance data, genotypic data, and location-specific environmental data associated with two or more training plants.

20. The system of embodiment 18, wherein the computing device further comprises:
one or more processors configured to perform operations prior to part (b)(2)(a), the operations comprising:
obtain at least one training data set comprising representations of genotypic data and location-specific environmental data associated with two or more training plants at one or more locations;
learn a relationship of the genotype by environment interactions with the phenotypic performance for a plurality of plant genotypes from the training data set representations and generate a predicted performance for a phenotype of interest for the one or more candidate plant genotypes to create a trained machine learning model.

21. (canceled)

22. (canceled)

23. (canceled)

24. The system of embodiment 18, wherein one or more servers, comprises representations of phenotypic performance data, genotypic data, and location-specific environmental data associated with two or more training plants.

25. The system of embodiment 18, wherein the computing device further comprises:
one or more processors configured to perform operations prior to part (b)(2)(a), the operations comprising:
obtain at least one training data set comprising representations of genotypic data and location-specific environmental data associated with two or more training plants at one or more locations;
learn a relationship among the training plant genotypes and location-specific environment interactions with phenotypic performance from the training data set and generate a predicted performance for a phenotype of interest for the one or more candidate plant genotypes to create a trained machine learning model.

26. (canceled)

27. (canceled)

28. A system comprising:
(a) one or more servers, wherein one of the servers comprises representations of genotypic data, location-specific environmental data, and spatial coordinate data associated with two or more candidate plant genotypes;
(b) a computing device communicatively coupled to the one or more servers, the computing device including:
(1) a memory; and
(2) one or more processors configured to perform an operation comprising: receive as input into a trained deep learning model representations of genotypic data for two or more candidate plant genotypes, location-specific environmental data, and spatial coordinate data as embedding vectors and generate a performance prediction for a phenotype of interest for one or more candidate plant genotypes as compared to a reference candidate plant genotype.

29. The system of claim 28, wherein one or more of the processors is configured to perform an operation comprising:
learn from the training data set to predict whether one or more of the candidate plant genotypes that are not the reference candidate plant genotype will perform better for the phenotype of interest compared to the reference candidate plant genotype.

30. The system of claim 20, 21, or 22, wherein one or more of the processors is configured to perform an operation comprising:
learn from the training data set to predict for at least one or more spatial units comprising a plant genotype a performance for a phenotype of interest compared to a predicted performance for the phenotype of interest for at least one spatial unit of a plant genotype; or
learn from the training data set to generate a predicted difference in the phenotype of interest between a first candidate plant and a second candidate plant at a certain location; or
learn from the training data set to generate a predicted performance difference, value, probability, or classification for a phenotype of interest for one or more candidate plant genotypes for one or more given locations, set of locations, or aggregated locations; or
learn from the training data set to generate a predicted performance for the phenotype of interest for one or more reference spatial units comprising at least one plant genotype.

31. (canceled)

32. The system of claim 18, the system further comprising one or more processors configured to perform an operation comprising:
generate a prediction as to the performance of the one or more candidate plant genotypes for the phenotype of interest at a certain location as compared to a reference candidate plant genotype at the same location or different location; or
generate a predicted relative performance value or a predicted difference for the one or more of the candidate plant genotypes for the phenotype of interest as compared to a reference candidate plant genotype's predicted performance for the phenotype; or
generate a prediction of the performance for each of the one or more of the selected candidate plant genotypes for the phenotype of interest, wherein the prediction is an average of the predicted performance value of that candidate plant genotype from the same or different locations and/or same or different environments.

33. The system of claim 18, wherein the phenotype of interest is yield, adjusted gross income (AGI), grain yield, yield gain, root lodging resistance, stalk lodging resistance, brittlesnap resistance, ear height, grain moisture, plant height, disease resistance, pest resistance, drought tolerance, cold tolerance, heat tolerance, salt tolerance, stress tolerance, herbicide tolerance, or flowering time.

34. (canceled)

35. (canceled)

36. The system of claim 18, wherein the location-specific environmental data comprises geographical location information, weather condition information and imagery, soil information, abiotic stress information, biotic stress information, plant growth stage information, plant developmental stage information, plant phenological stage information, planting conditions, or combinations thereof.

37. The system of claim 18, the system further comprising one or more processors configured to perform an operation comprising: direct at least one or more of the candidate plants into a breeding pipeline.

38. The system of claim 18, the system further comprising
a data structure comprising plant genotypes available for use in training a machine learning model or deep learning model, location-specific environmental data and/or spatial coordinate data, and associated phenotypic performance data for use in deriving target outputs, or a data structure comprising candidate plant genotypes available for use in predicting performance for the phenotype of interest by a machine learning model or deep learning model, location-specific environmental data, and/or spatial coordinate data.
